(19) United States
(12) Patent Application Publication
(10) Pub. No.: US 2026/0347355 A1
(43) Pub. Date: Nov. 5, 2026

(54) COMPOUNDS

(71) Applicant: Hoffmann-La Roche Inc., Little Falls, NJ (US)

(72) Inventors: Lea Aurelie BOUCHE, Basel-City (CH); Wolfgang GUBA, Müllheim (DE); Sabrina HERR, Horgen (CH); Georg Stefan JAESCHKE, Basel-City (CH); Stefanie Katharina MESCH, Basel-City (CH); Christian SCHNIDER, Biel-Benken (CH); Sandra STEINER, Sursee (CH)

(73) Assignee: Hoffmann-La Roche Inc., Little Falls, NJ (US)

(21) Appl. No.: 19/544,249

(22) Filed: Feb. 19, 2026

Related U.S. Application Data
(63) Continuation of application No. PCT/EP2024/074103, filed on Aug. 29, 2024.

(30) Foreign Application Priority Data
Aug. 31, 2023 (EP) 23194409.1

(51) Int. Cl. C07D 471/04; A61K 31/5025

(52) U.S. Cl. CPC C07D 471/04; A61K 31/5025

(57) ABSTRACT

The invention relates to novel compounds having the general formula I, wherein Rₓ and R_{y} are as described herein, composition including the compounds and methods of using the compounds.

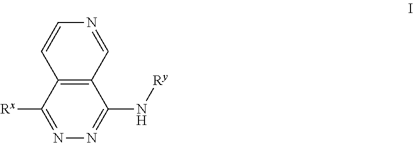

### FIELD OF THE INVENTION

The present invention relates to organic compounds useful for therapy and/or prophylaxis in a mammal, and in particular to compounds that modulate NLRP3 inhibition.

The present invention provides novel compounds of formula I

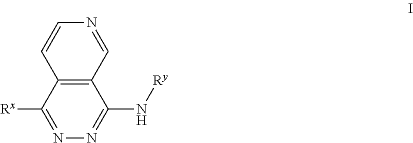

wherein,
   R^{x} is selected from

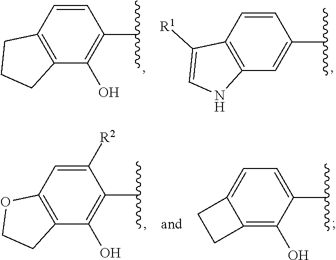

R¹ is H or halo;
R² is H, methyl, —CH₂OCH₃, or —CH₂OH;
R^{y} is selected from

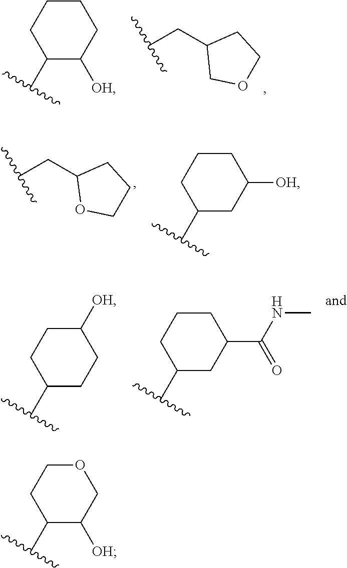

and pharmaceutically acceptable salts thereof.

Furthermore, the invention includes all racemic mixtures, all their corresponding enantiomers, diastereomers and/or optical isomers.

### BACKGROUND OF THE INVENTION

The NOD-like receptor (NLR) family, pyrin domain-containing protein 3 (NLRP3) inflammasome is a component of the inflammatory process, and its aberrant activity is pathogenic in inherited disorders such as cryopyrin-associated periodic syndromes (CAPS) and complex diseases such as multiple sclerosis, type 2 diabetes, Alzheimer's disease and atherosclerosis.

NLRP3 is an intracellular signaling molecule that senses many pathogen-derived, environmental and host-derived factors. Upon activation, NLRP3 binds to apoptosis-associated speck-like protein containing a caspase activation and recruitment domain (ASC). ASC then polymerises to form a large aggregate known as an ASC speck. Polymerised ASC in turn interacts with the cysteine protease caspase-1 to form a complex termed the inflammasome. This results in the activation of caspase-1, which cleaves the precursor forms of the proinflammatory cytokines IL-1β and IL-18 (termed pro-IL-1β and pro-IL-18 respectively) to thereby activate these cytokines. Caspase-1 also mediates a type of inflammatory cell death known as pyroptosis. The ASC speck can also recruit and activate caspase-8, which can process pro-IL-1β and pro-IL-18 and trigger apoptotic cell death.

Caspase-1 cleaves pro-IL-1β and pro-IL-18 to their active forms, which are secreted from the cell. Active caspase-1 also cleaves gasdermin-D to trigger pyroptosis. Through its control of the pyroptotic cell death pathway, caspase-1 also mediates the release of alarmin molecules such as IL-33 and high mobility group box 1 protein (HMGB1). Caspase-1 also cleaves intracellular IL-1R2 resulting in its degradation and allowing the release of IL-1α. In human cells caspase-1 may also control the processing and secretion of IL-37. A number of other caspase-1 substrates such as components of the cytoskeleton and glycolysis pathway may contribute to caspase-1-dependent inflammation.

NLRP3-dependent ASC specks are released into the extracellular environment where they can activate caspase-1, induce processing of caspase-1 substrates and propagate inflammation.

Active cytokines derived from NLRP3 inflammasome activation are important drivers of inflammation and interact with other cytokine pathways to shape the immune response to infection and injury. For example, IL-1β signalling induces the secretion of the pro-inflammatory cytokines IL-6 and TNF. IL-1β and IL-18 synergise with IL-23 to induce IL-17 production by memory CD4 Th17 cells and by γδ T cells in the absence of T cell receptor engagement. IL-18 and IL-12 also synergise to induce IFN-γ production from memory T cells and NK cells driving a Th1 response.

The inherited CAPS diseases Muckle-Wells syndrome (MWS), familial cold autoinflammatory syndrome (FCAS) and neonatal-onset multisystem inflammatory disease (NOMID) are caused by gain-of-function mutations in NLRP3, thus defining NLRP3 as a critical component of the inflammatory process. NLRP3 has also been implicated in the pathogenesis of a number of complex diseases, notably including metabolic disorders such as type 2 diabetes, atherosclerosis, obesity and gout.

A role for NLRP3 in diseases of the central nervous system is emerging, and lung diseases have also been shown to be influenced by NLRP3. NLRP3 has also been suggested to have a role in a number of central nervous system conditions, including Parkinson's disease (PD), Alzheimer's disease (AD), dementia, Huntington's disease, cerebral malaria, brain injury from pneumococcal meningitis (Walsh et al., Nature Reviews, 15: 84-97, 2014, and Dempsey et al. Brain. Behav. Immun. 201761: 306-316). NLRP3 has also been shown to play a role in a number of lung diseases including chronic obstructive pulmonary disorder (COPD), asthma (including steroid-resistant asthma), asbestosis, and silicosis (De Nardo et al., Am. J. Pathol., 184: 42-54, 2014 and Kim et al. Am J Respir Crit Care Med. 2017 196(3): 283-97). Furthermore, NLRP3 has a role in the development of liver disease, kidney disease and aging. Many of these associations were defined using Nlrp3^{-/-} mice, but there have also been insights into the specific activation of NLRP3 in these diseases. In type 2 diabetes mellitus (T2D), the deposition of islet amyloid polypeptide in the pancreas activates NLRP3 and IL-1β signalling, resulting in cell death and inflammation.

Several small molecules have been shown to inhibit the NLRP3 inflammasome. Glyburide inhibits IL-1β production at micromolar concentrations in response to the activation of NLRP3 but not NLRC4 or NLRP1. Other previously characterised weak NLRP3 inhibitors include parthenolide, 3,4-methylenedioxy-β-nitrostyrene and dimethyl sulfoxide (DMSO), although these agents have limited potency and are nonspecific.

Current treatments for NLRP3-related diseases include biologic agents that target IL-I. These are the recombinant IL-I receptor antagonist anakinra, the neutralizing IL-1β antibody canakinumab and the soluble decoy IL-1 receptor rilonacept. These approaches have proven successful in the treatment of CAPS, and these biologic agents have been used in clinical trials for other IL-1β-associated diseases. There is a need to provide compounds with improved pharmacological and/or physiological and/or physicochemical properties and/or those that provide a useful alternative to known compounds.

### SUMMARY OF THE INVENTION

The present invention provides novel compounds of formula I

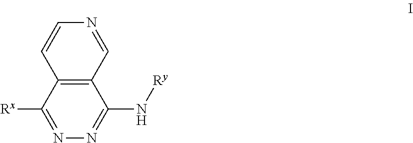

wherein,
   R^{x} is selected from

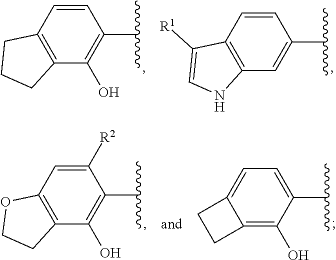

R¹ is H or halo;
R² is H, methyl, —CH₂OCH₃, or —CH₂OH;
R^{y} is selected from

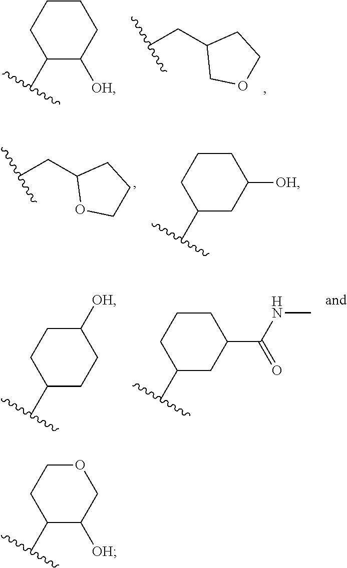

and pharmaceutically acceptable salts thereof.

The term “halogen”, “halide” and “halo” are used interchangeably herein and denote fluoro, chloro, bromo or iodo. Examples of “halo” include fluoro.

The term “hydroxy” denotes a —OH group.

The term “pharmaceutically acceptable salts” refers to those salts which retain the biological effectiveness and properties of the free bases or free acids, which are not biologically or otherwise undesirable. The salts are formed with inorganic acids such as trifluoroacetic acid, hydrochloric acid, hydrobromic acid, sulfuric acid, nitric acid, phosphoric acid, particularly hydrochloric acid, and organic acids such as formic acid, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, N-acetyleystein. In addition these salts may be prepared from addition of an inorganic base or an organic base to the free acid. Salts derived from an inorganic base include, but are not limited to, the sodium, potassium, lithium, ammonium, calcium, magnesium salts. Salts derived from organic bases include, but are not limited to salts of primary, secondary, and tertiary amines, substituted amines including naturally occurring substituted amines, cyclic amines and basic ion exchange resins, such as isopropylamine, trimethylamine, diethylamine, triethylamine, tripropylamine, ethanolamine, lysine, arginine, N-ethylpiperidine, piperidine, polyamine resins. The compound of formula I can also be present in the form of zwitterions. Particularly preferred pharmaceutically acceptable salts of compounds of formula I are the salts formed with formic acid and the salts formed with hydrochloric acid yielding a hydrochloride, dihydrochloride or trihydrochloride salt.

The abbreviation uM means microMolar and is equivalent to the symbol μM.

The abbreviation uL means microliter and is equivalent to the symbol μL.

The abbreviation ug means microgram and is equivalent to the symbol μg.

The compounds of formula I can contain several asymmetric centers and can be present in the form of optically pure enantiomers, mixtures of enantiomers such as, for example, racemates, optically pure diastereoisomers, mixtures of diastereoisomers, diastereoisomeric racemates or mixtures of diastereoisomeric racemates.

According to the Cahn-Ingold-Prelog Convention the asymmetric carbon atom can be of the “R” or “S” configuration.

Also an embodiment of the present invention provides compounds according to formula I as described herein and pharmaceutically acceptable salts or esters thereof, in particular compounds according to formula I as described herein and pharmaceutically acceptable salts thereof, more particularly compounds according to formula I as described herein.

An embodiment of the present invention provides compounds according to formula I as described herein, wherein Rₓ is

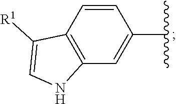

### and R¹ is H or Halo.

An embodiment of the present invention provides compounds according to formula I as described herein, wherein R² is methyl or —CH₂OCH₃.

An embodiment of the present invention provides compounds according to formula I as described herein, wherein R^{y} is selected from

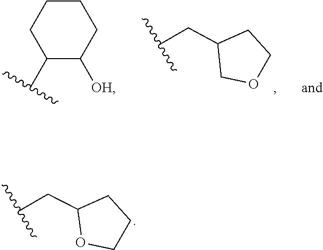 [SMILES: *C1CCCCC1O ; *CC1CCOC1 ; *CC1CCCO1]

An embodiment of the present invention provides compounds according to formula I as described herein, wherein R^{y} is selected from

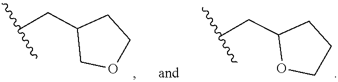 [SMILES: *CC1CCOC1 ; *CC1CCCO1]

An embodiment of the present invention provides compounds according to formula I as described herein, wherein R^{y} is

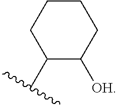 [SMILES: *C1CCCCC1O]

An embodiment of the present invention provides compounds according to formula I as described herein, wherein R^{y} is selected from

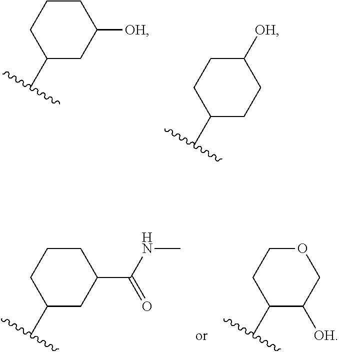 [SMILES: *C1CCCC(O)C1 ; *C1CCC(O)CC1 ; *C1CCCC(C(=O)NC)C1 ; *C1CCOCC1O]

An embodiment of the present invention provides compounds according to formula I as described herein, wherein R^{y} is

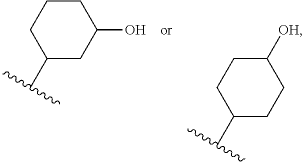 [SMILES: *C1CCCC(O)C1 ; *C1CCC(O)CC1]

An embodiment of the present invention provides compounds according to formula I as described herein, wherein R^{y} is

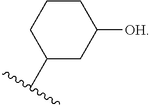 [SMILES: *C1CCCC(O)C1]

An embodiment of the present invention provides compounds according to formula I as described herein, wherein R^{y} is

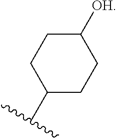 [SMILES: *C1CCC(O)CC1]

An embodiment of the present invention provides compounds according to formula I as described herein, wherein R^{y} is

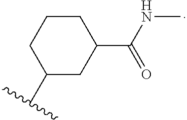 [SMILES: *C1CCCC(C(=O)NC)C1]

An embodiment of the present invention provides compounds according to formula I as described herein, wherein R^{y} is

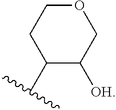 [SMILES: *C1CCOCC1O]

An embodiment of the present invention provides compounds according to formula I as described herein, wherein
R^{x} is selected from

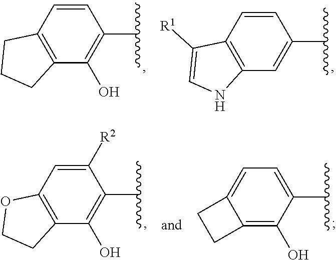

R¹ is H or halo;
R² is H, methyl or —CH₂OCH₃;
R^{y} is selected from

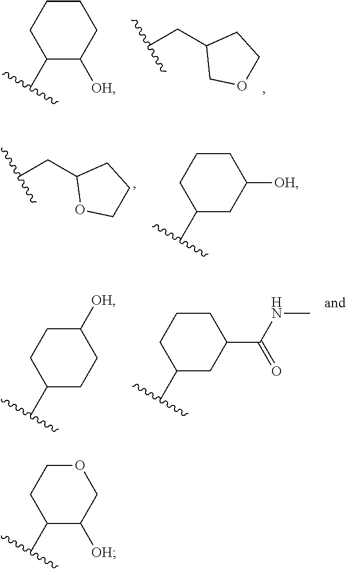

and pharmaceutically acceptable salts thereof.

An embodiment of the present invention provides compounds according to formula I as described herein, wherein
R^{x} is

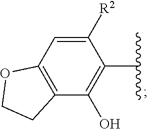

R² is H, methyl or —CH₂OCH₃;
R^{y} is selected from

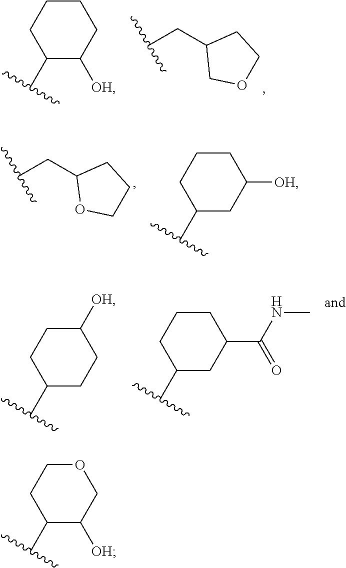

and pharmaceutically acceptable salts thereof.

An embodiment of the present invention provides compounds according to formula I as described herein, wherein
R^{x} is

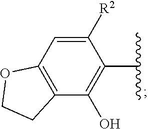

R² is H or —CH₂OCH₃;
R^{y} is selected from

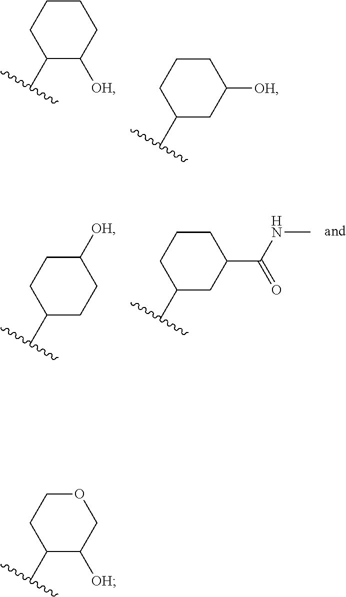 [SMILES: *C1CCCCC1O ; *C1CCCC(O)C1 ; *C1CCC(O)CC1 ; *C1CCCC(C(=O)NC)C1 ; *C1CCOCC1O]

and pharmaceutically acceptable salts thereof.

An embodiment of the present invention provides compounds according to formula I as described herein, wherein
R^{x} is

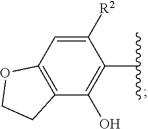

R² is —CH₂OCH₃;
R^{y} is selected from

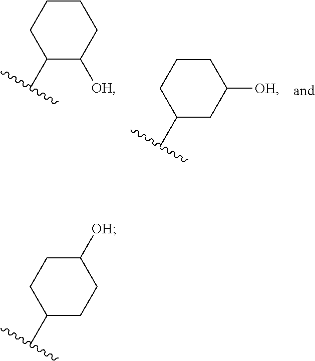 [SMILES: *C1CCCCC1O ; *C1CCCC(O)C1 ; *C1CCC(O)CC1]

and pharmaceutically acceptable salts thereof.

An embodiment of the present invention provides compounds according to formula I as described herein, wherein
R^{x} is

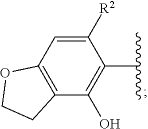

R² is H;
R^{y} is selected from

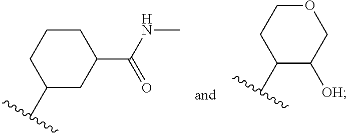 [SMILES: *C1CCCC(C(=O)NC)C1 ; *C1CCOCC1O]

and pharmaceutically acceptable salts thereof.

Particular examples of compounds of formula I as described herein are selected from
5-[4-[[rac-(1R,2R)-2-Hydroxycyclohexyl]aamino]pyrido[3,4-d]pyridazin-1-yl]indan-4-ol;
5-[4-[[rel-(1R,2R)-2-hydroxycyclohexyl]amino]pyrido[3,4-d]pyridazin-1-yl]indan-4-ol;
5-[4-[[rel-(1S,2S)-2-hydroxycyclohexyl]amino]pyrido[3,4-d]pyridazin-1-yl]indan-4-ol;
5-[4-[[rac-(1R,2R)-2-hydroxycyclohexyl]amino]pyrido[3,4-d]pyridazin-1-yl]-2,3-dihydrobenzofuran-4-ol;
5-[4-[[rel-(1R,2R)-2-Hydroxycyclohexyl]amino]pyrido[3,4-d]pyridazin-1-yl]-2,3-dihydrobenzofuran-4-ol;
5-[4-[[rel-(1S,2S)-2-Hydroxycyclohexyl]amino]pyrido[3,4-d]pyridazin-1-yl]-2,3-dihydrobenzofuran-4-ol;
rac-(1R,2R)-2-[[1-(1H-indol-6-yl)pyrido[3,4-d]pyridazin-4-yl]amino]cyclohexanol;
1-(3-Fluoro-1H-indol-6-yl)-N-[[rel-(3R)-tetrahydrofuran-3-yl]methyl]pyrido[3,4-d]pyridazin-4-amine;
1-(3-Fluoro-1H-indol-6-yl)-N-[[rel-(3S)-tetrahydrofuran-3-yl]methyl]pyrido[3,4-d]pyridazin-4-amine;
5-[4-[[rac-(3S)-Tetrahydrofuran-3-yl]methylamino]pyrido[3,4-d]pyridazin-1-yl]-2,3-dihydrobenzofuran-4-ol;
5-[4-[[rel-(3R)-Tetrahydrofuran-3-yl]methylamino]pyrido[3,4-d]pyridazin-1-yl]-2,3-dihydrobenzofuran-4-ol;
5-[4-[[rel-(3S)-Tetrahydrofuran-3-yl]methylamino]pyrido[3,4-d]pyridazin-1-yl]-2,3-dihydrobenzofuran-4-ol;
5-[4-[[rel-(2R)-Tetrahydrofuran-2-yl]methylamino]pyrido[3,4-d]pyridazin-1-yl]-2,3-dihydrobenzofuran-4-ol;
5-[4-[[rel-(2S)-Tetrahydrofuran-2-yl]methylamino]pyrido[3,4-d]pyridazin-1-yl]-2,3-dihydrobenzofuran-4-ol;
3-[4-[[rac-(1R,2R)-2-Hydroxycyclohexyl]amino]pyrido[3,4-d]pyridazin-1-yl]bicyclo[4.2.0]octa-1,3,5-trien-2-ol;
   and pharmaceutically acceptable salts thereof.

Other examples of compounds of formula I as described herein are selected from
5-[4-[[(1R,2R)-2-Hydroxycyclohexyl]amino]pyrido[3,4-d]pyridazin-1-yl]-6-(methoxymethyl)-2,3-dihydrobenzofuran-4-ol;
trans 5-[4-[(4-Hydroxycyclohexyl)amino]pyrido[3,4-d]pyridazin-1-yl]-2,3-dihydrobenzofuran-4-ol;
5-[4-[[(IR,2R)-2-Hydroxycyclohexyl]amino]pyrido[3,4-d]pyridazin-1-yl]-6-methyl-2,3-dihydrobenzofuran-4-ol;
cis 5-[4-[(4-Hydroxycyclohexyl)amino]pyrido[3,4-d]pyridazin-1-yl]-2,3-dihydrobenzofuran-4-ol;
5-[4-[[(1R,3S)-3-Hydroxycyclohexyl]amino]pyrido[3,4-d]pyridazin-1-yl]-2,3-dihydrobenzofuran-4-ol;
(1S,3R)-3-[[1-(4-Hydroxy-2,3-dihydrobenzofuran-5-yl)pyrido[3,4-d]pyridazin-4-yl]amino]-N-methyl-cyclohexanecarboxamide;
5-[4-[[(3R,4S)-3-Hydroxytetrahydropyran-4-yl]amino]pyrido[3,4-d]pyridazin-1-yl]-2,3-dihydrobenzofuran-4-ol;
5-[4-[[(3S,4R)-3-Hydroxytetrahydropyran-4-yl]amino]pyrido[3,4-d]pyridazin-1-yl]-2,3-dihydrobenzofuran-4-ol;
   and pharmaceutically acceptable salts thereof.

Another embodiment of the invention provides a pharmaceutical composition or medicament containing a compound of the invention and a therapeutically inert carrier, diluent or excipient, as well as a method of using the compounds of the invention to prepare such composition and medicament. In one example, the compound of formula I may be formulated by mixing at ambient temperature at the appropriate pH, and at the desired degree of purity, with physiologically acceptable carriers, i.e., carriers that are non-toxic to recipients at the dosages and concentrations employed into a galenical administration form. The pH of the formulation depends mainly on the particular use and the concentration of compound, but preferably ranges anywhere from about 3 to about 8. In one example, a compound of formula I is formulated in an acetate buffer, at pH 5. In another embodiment, the compound of formula I is sterile. The compound may be stored, for example, as a solid or amorphous composition, as a lyophilized formulation or as an aqueous solution.

Compositions are formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners.

The compounds of the invention may be administered by any suitable means, including oral, topical (including buccal and sublingual), rectal, vaginal, transdermal, parenteral, subcutaneous, intraperitoneal, intrapulmonary, intradermal, intrathecal and epidural and intranasal, and, if desired for local treatment, intralesional administration. Parenteral infusions include intramuscular, intravenous, intraarterial, intraperitoneal, or subcutaneous administration.

The compounds of the present invention may be administered in any convenient administrative form, e.g., tablets, powders, capsules, solutions, dispersions, suspensions, syrups, sprays, suppositories, gels, emulsions, patches, etc. Such compositions may contain components conventional in pharmaceutical preparations, e.g., diluents, carriers, pH modifiers, sweeteners, bulking agents, and further active agents.

A typical formulation is prepared by mixing a compound of the present invention and a carrier or excipient. Suitable carriers and excipients are well known to those skilled in the art and are described in detail in, e.g., Ansel, Howard C., et al., Ansel's Pharmaceutical Dosage Forms and Drug Delivery Systems. Philadelphia: Lippincott, Williams & Wilkins, 2004; Gennaro, Alfonso R., et al. Remington: The Science and Practice of Pharmacy. Philadelphia: Lippincott, Williams & Wilkins, 2000; and Rowe, Raymond C. Handbook of Pharmaceutical Excipients. Chicago, Pharmaceutical Press, 2005. The formulations may also include one or more buffers, stabilizing agents, surfactants, wetting agents, lubricating agents, emulsifiers, suspending agents, preservatives, antioxidants, opaquing agents, glidants, processing aids, colorants, sweeteners, perfuming agents, flavoring agents, diluents and other known additives to provide an elegant presentation of the drug (i.e., a compound of the present invention or pharmaceutical composition thereof) or aid in the manufacturing of the pharmaceutical product (i.e., medicament).

The compounds of formula I and their pharmaceutically acceptable salts can be processed with pharmaceutically inert, inorganic or organic adjuvants for the production of tablets, coated tablets, dragées, hard gelatin capsules, injection solutions or topical formulations Lactose, corn starch or derivatives thereof, tale, stearic acid or its salts etc. can be used, for example, as such adjuvants for tablets, dragées and hard gelatin capsules.

Suitable adjuvants for soft gelatin capsules, are, for example, vegetable oils, waxes, fats, semi-solid substances and liquid polyols, etc.

Suitable adjuvants for the production of solutions and syrups are, for example, water, polyols, saccharose, invert sugar, glucose, etc.

Suitable adjuvants for injection solutions are, for example, water, alcohols, polyols, glycerol, vegetable oils, etc.

Suitable adjuvants for suppositories are, for example, natural or hardened oils, waxes, fats, semi-solid or liquid polyols, etc.

Suitable adjuvants for topical ocular formulations are, for example, cyclodextrins, mannitol or many other carriers and excipients known in the art.

Moreover, the pharmaceutical preparations can contain preservatives, solubilizers, viscosity-increasing substances, stabilizers, wetting agents, emulsifiers, sweeteners, colorants, flavorants, salts for varying the osmotic pressure, buffers, masking agents or antioxidants. They can also contain still other therapeutically valuable substances.

The dosage can vary in wide limits and will, of course, be fitted to the individual requirements in each particular case. In general, in the case of oral administration a daily dosage of about 0.1 mg to 20 mg per kg body weight, preferably about 0.5 mg to 4 mg per kg body weight (e.g. about 300 mg per person), divided into preferably 1-3 individual doses, which can consist, for example, of the same amounts, should it be appropriate. In the case of topical administration, the formulation can contain 0.001% to 15% by weight of medicament and the required dose, which can be between 0.1 and 25 mg in can be administered either by single dose per day or per week, or by multiple doses (2 to 4) per day, or by multiple doses per week It will, however, be clear that the upper or lower limit given herein can be exceeded when this is shown to be indicated.

An embodiment of the present invention is a compound according to formula I as described herein for use as a therapeutically active substance.

An embodiment of the present invention is a compound according to formula I as described herein for use in the treatment or prevention of a disease, disorder or condition, wherein the disease, disorder or condition is responsive to NLRP3 inhibition.

An embodiment of the present invention is a compound according to formula I as described herein for the treatment or prophylaxis of a disease, disorder or condition, wherein the disorder or condition is responsive to NLRP3 inhibition.

As used herein, the term “NLRP3 inhibition” refers to the complete or partial reduction in the level of activity of NLRP3 and includes, for example, the inhibition of active NLRP3 and/or the inhibition of activation of NLRP3.

There is evidence for a role of NLRP3-induced IL-1 and IL-18 in the inflammatory responses occurring in connection with, or as a result of, a multitude of different disorders (Menu et al., Clinical and Experimental Immunology, 166: 1-15, 2011; Strowig et al., Nature, 481: 278-286, 2012).

In one embodiment, the disease, disorder or condition is selected from:
(i) inflammation;
(ii) an auto-immune disease;
(iii) cancer;
(iv) an infection;
(v) a central nervous system disease;
(vi) a metabolic disease;
(vii) a cardiovascular disease;
(viii) a respiratory disease;
(ix) a liver disease;
(x) a renal disease;
(xi) an ocular disease;
(xii) a skin disease;
(xiii) a lymphatic condition;
(xiv) a psychological disorder;
(xv) graft versus host disease;
(xvi) allodynia;
(xvii) a condition associated with diabetes; and
(xviii) any disease where an individual has been determined to carry a germline or somatic non-silent mutation in NLRP3

In another embodiment, the disease, disorder or condition is selected from:
(i) cancer;
(ii) an infection;
(iii) a central nervous system disease;
(iv) a cardiovascular disease;
(v) a liver disease;
(vi) an ocular disease; and
(vii) a skin disease.

In a further typical embodiment of the invention, the disease, disorder or condition is inflammation. Examples of inflammation that may be treated or prevented include inflammatory responses occurring in connection with, or as a result of:
(i) a skin condition such as contact hypersensitivity, bullous pemphigoid, sunburn, psoriasis, atopical dermatitis, contact dermatitis, allergic contact dermatitis, seborrhoetic dermatitis, lichen planus, scleroderma, pemphigus, epidermolysis bullosa, urticaria, erythemas, or alopecia;
(ii) a joint condition such as osteoarthritis, systemic juvenile idiopathic arthritis, adult-onset Still's disease, relapsing polychondritis, rheumatoid arthritis, juvenile chronic arthritis, gout, or a seronegative spondyloarthropathy (e.g. ankylosing spondylitis, psoriatic arthritis or Reiter's disease);
(iii) a muscular condition such as polymyositis or myasthenia gravis;
(iv) a gastrointestinal tract condition such as inflammatory bowel disease (including Crohn's disease and ulcerative colitis), colitis, gastric ulcer, Coeliac disease, proctitis, pancreatitis, eosinopilic gastro-enteritis, mastocytosis, antiphospholipid syndrome, or a food-related allergy which may have effects remote from the gut (e.g., migraine, rhinitis or eczema);
(v) a respiratory system condition such as chronic obstructive pulmonary disease (COPD), asthma (including eosinophilic, bronchial, allergic, intrinsic, extrinsic or dust asthma, and particularly chronic or inveterate asthma, such as late asthma and airways hyper-responsiveness), bronchitis, rhinitis (including acute rhinitis, allergic rhinitis, atrophic rhinitis, chronic rhinitis, rhinitis caseosa, hypertrophic rhinitis, rhinitis pumlenta, rhinitis sicca, rhinitis medicamentosa, membranous rhinitis, seasonal rhinitis e.g. hay fever, and vasomotor rhinitis), sinusitis, idiopathic pulmonary fibrosis (IPF), sarcoidosis, farmer's lung, silicosis, asbestosis, volcanic ash induced inflammation, adult respiratory distress syndrome, hypersensitivity pneumonitis, or idiopathic interstitial pneumonia;
(vi) a vascular condition such as atherosclerosis, Behcet's disease, vasculitides, or Wegener's granulomatosis;
(vii) an autoimmune condition such as systemic lupus erythematosus, Sjdgren's syndrome, systemic sclerosis, Hashimoto's thyroiditis, type I diabetes, idiopathic thrombocytopenia purpura, or Graves disease;
(viii) an ocular condition such as uveitis, allergic conjunctivitis, or vernal conjunctivitis;
(ix) a nervous condition such as multiple sclerosis or encephalomyelitis;
(x) an infection or infection-related condition, such as Acquired Immunodeficiency Syndrome (AIDS), acute or chronic bacterial infection, acute or chronic parasitic infection, acute or chronic viral infection, acute or chronic fungal infection, meningitis, hepatitis (A, B or C, or other viral hepatitis), peritonitis, pneumonia, epiglottitis, malaria, dengue hemorrhagic fever, leishmaniasis, streptococcal myositis, Mycobacterium tuberculosis (including Mycobacterium tuberculosis and HIV co-infection), Mycobacterium avium intracellulare, Pneumocystis carinii pneumonia, orchitis/epidydimitis, legionella, Lyme disease, influenza A, Epstein-Barr virus infection, viral encephalitis/aseptic meningitis, or pelvic inflammatory disease;
(xi) a renal condition such as mesangial proliferative glomerulonephritis, nephrotic syndrome, nephritis, glomerular nephritis, obesity related glomerulopathy, acute renal failure, acute kidney injury, uremia, nephritic syndrome, kidney fibrosis including chronic crystal nephropathy, or renal hypertension;
(xii) a lymphatic condition such as Castleman's disease;
(xiii) a condition of, or involving, the immune system, such as hyper IgE syndrome, lepromatous leprosy, familial hemophagocytic lymphohistiocytosis, or graft versus host disease;
(xiv) a hepatic condition such as chronic active hepatitis, non-alcoholic steatohepatitis (NASH), alcohol-induced hepatitis, non-alcoholic fatty liver disease (NAFLD), alcoholic fatty liver disease (AFLD), alcoholic steatohepatitis (ASH), primary biliary cirrhosis, fulminant hepatitis, liver fibrosis, or liver failure;
(xv) a cancer, including those cancers listed above;
(xvi) a burn, wound, trauma, haemorrhage or stroke;
(xvii) radiation exposure;
(xviii) a metabolic disease such as type 2 diabetes (T2D), atherosclerosis, obesity, gout or pseudo-gout; and/or
(xix) pain such as inflammatory hyperalgesia, pelvic pain, allodynia, neuropathic pain, or cancer-induced bone pain.

An embodiment of the present invention is a compound according to formula I as described herein for the treatment or prophylaxis of a disease, disorder or condition selected from:
inflammation;
an auto-immune disease;
cancer;
an infection;
a central nervous system disease;
a metabolic disease;
a cardiovascular disease;
a respiratory disease;
a liver disease;
a renal disease;
an ocular disease;
a skin disease;
a lymphatic condition;
a psychological disorder;
graft versus host disease;
allodynia;
a condition associated with diabetes; and
any disease where an individual has been determined to carry a germline or somatic non-silent mutation in NLRP3.

An embodiment of the present invention is the use of a compound according to formula I as described herein in the treatment or prophylaxis of a disease, disorder or condition, wherein the disease, disorder or condition is responsive to NLRP3 inhibition.

An embodiment of the present invention is the use of a compound according to formula I as described herein in the treatment or prophylaxis of a disease, disorder or condition selected from Alzheimer's disease and Parkinson's disease.

An embodiment of the present invention is the use a compound according to formula I as described herein for use in the treatment or prophylaxis of a disease, disorder or condition selected from Asthma and COPD.

An embodiment of the present invention is a compound according to formula I as described herein for the treatment or prophylaxis of a disease, disorder or condition selected from Alzheimer's disease and Parkinson's disease.

An embodiment of the present invention is a compound according to formula I as described herein for the treatment or prophylaxis of a disease, disorder or condition selected from Asthma and COPD.

An embodiment of the present invention is the use of a compound according to formula I as described herein for preparation of a medicament for the treatment or prophylaxis of a disease, disorder or condition selected from Alzheimer's disease and Parkinson's disease.

An embodiment of the present invention is the use of a compound according to formula I as described herein for the preparation of a medicament for the treatment or prophylaxis of a disease, disorder or condition selected from Asthma and COPD.

An embodiment of the present invention is a method of treatment or prophylaxis of a disease, disorder or condition selected from Alzheimer's disease and Parkinson's disease, which method comprises administering an effective amount of a compound according to formula I as described herein.

An embodiment of the present invention is a method of treatment or prophylaxis of a disease, disorder or condition selected from Asthma and COPD, which method comprises administering an effective amount of a compound according to formula I as described herein.

An embodiment of the present invention relates to a method of inhibiting NLRP3, which method comprises administering an effective amount of a compound according to formula I as described herein.

Also an embodiment of the present invention are compounds of formula I as described herein, when manufactured according to any one of the described processes.

An embodiment of the present invention is a pharmaceutical composition comprising a compound according to formula I as described herein and a therapeutically inert carrier.

### Assay Procedures

### NLRP3 and Pyroptosis

It is well established that the activation of NLRP3 leads to cell pyroptosis and this feature plays an important part in the manifestation of clinical disease (Yan-gang Liu et al., Cell Death & Disease, 2017, 8(2), e2579; Alexander Wree et al., Hepatology, 2014, 59(3), 898-910; Alex Baldwin et al., Journal of Medicinal Chemistry, 2016, 59(5), 1691-1710; Ema Ozaki et al., Journal of Inflammation Research, 2015, 8, 15-27; Zhen Xie & Gang Zhao, Neuroimmunology Neuroinflammation, 2014, 1(2), 60-65; Mattia Cocco et al., Journal of Medicinal Chemistry, 2014, 57(24), 10366-10382; T. Satoh et al., Cell Death & Disease, 2013, 4, e644). Therefore, it is anticipated that inhibitors of NLRP3 will block pyroptosis, as well as the release of pro-inflammatory cytokines (e.g. IL-1β) from the cell.

### THP-1 Cells: Culture and Preparation

THP-1 cells (ATCC #TIB-202) were grown in RPMI containing L-glutamine (Gibco #11835) supplemented with 1 mM sodium pyruvate (Sigma #S8636) and penicillin (100units/ml)/streptomycin (0.1 mg/ml) (Sigma #P4333) in 10% Fetal Bovine Serum (FBS) (Sigma #F0804). The cells were routinely passaged and grown to confluency (~10⁶cells/ml). On the day of the experiment, THP-1 cells were harvested and resuspended into RPMI medium (without FBS). The cells were then counted and viability (>90%) checked by Trypan blue (Sigma #T8154). Appropriate dilutions were made to give a concentration of 625,000cells/ml. To this diluted cell solution was added LPS (Sigma #L4524) to give a 1 μg/ml Final Assay Concentration (FAC). 40 μl of the final preparation was aliquoted into each well of a 96-well plate. The plate thus prepared was used for compound screening.

### THP-1 Cells Pyroptosis Assay

The following method step-by-step assay was followed for compound screening.

Seed THP-1 cells (25,000cells/well) containing 1.0 μg/ml LPS in 40 μl of RPMI medium (without FBS) in 96-well, black walled, clear bottom cell culture plates coated with poly-D-lysine (VWR #734-0317)

Add 5 μl compound (8 points half-log dilution, with 10 μM top dose) or vehicle (DMSO 0.1% FAC) to the appropriate wells

Incubate for 3 hours at 37 °C, 5% CO₂

Add 5 μl nigericin (Sigma #N7143) (FAC 5 μM) to all wells

Incubate for 1 hr at 37 °C, 5% CO₂

At the end of the incubation period, spin plates at 300×g for 3 mins and remove supernatant

Then add 50 μl of resazurin (Sigma #R7017) (FAC 100 μM resazurin in RPMI medium without FBS) and incubate plates for a further 1-2 hours at 37 °C and 5% CO₂

Plates were read in an Envision reader at Ex 560 nm and Em 590 nm

IC₅₀ data is fitted to a non-linear regression equation (log inhibitor vs response-variable slope 4-parameters)

The results of the pyroptosis assay are summarised in Table 1 below as THP IC₅₀.

### Human Whole Blood IL-1β Release Assay

For systemic delivery, the ability to inhibit NLRP3 when the compounds are present within the bloodstream is of great importance. For this reason, the NLRP3 inhibitory activity of a number of compounds in human whole blood was investigated in accordance with the following protocol.

Human whole blood in Li-heparin tubes was obtained from healthy donors from a volunteer donor panel. Plate out 80 μl of whole blood containing 1 μg/ml of LPS in 96-well, clear bottom cell culture plate (Corning #3585)

Add 10 μl compound (8 points half-log dilution with 10 μM top dose) or vehicle (DMSO 0.1% FAC) to the appropriate wells

Incubate for 3 hours at 37 °C, 5% CO₂

Add 10 μl nigericin (Sigma #N7143) (10 μM FAC) to all wells

Incubate for 1 hr at 37 °C, 5% CO₂

At the end of the incubation period, spin plates at 300×g for 5 mins to pellet cells and remove 20 μl of supernatant and add to 96-well v-bottom plates for IL-1β analysis (note: these plates containing the supernatants can be stored at −80 °C to be analysed at a later date)

IL-1β was measured according to the manufacturer protocol (Perkin Elmer-AlphaLisa IL-1 Kit AL220F-5000)

IC₅₀ data is fitted to a non-linear regression equation (log inhibitor vs response-variable slope 4-parameters)

The results of the human whole blood assay are summarised in Table 1 below as HWB IC₅₀.

### Microsomal Stability:

Incubations of test compounds at 1 μM in microsomes (0.5 mg/mL) plus cofactor NADPH are performed in 96 well plates at 37 °C on a TECAN (Tecan Group Ltd, Switzerland) automated liquid handling system. After a 10 minutes pre-incubation step of the test compound with the microsomes, the enzymatic reaction is started by the addition of cofactors. At 1, 3, 6, 9, 15, 25, 35 and 45 minutes, aliquots of the incubations are removed and quenched with 1:3 (v/v) acetonitrile containing internal standard. Samples are then cooled and centrifuged before analysis of the supernatant by LC-MS/MS 2.

### Metabolic Stability in Hepatocytes:

### Assay Descriptions:

Biological materials. Cryopreserved hepatocytes [mouse, rat, rabbit, monkey and human (male and female; mixed)] are obtained. Viability of hepatocytes after reconstitution is at least 80% throughout the study. Ready-to-use rat/human HepatoPac® cultures [long-term hepatocyte co-cultures; pooled (n=5 for male and n=5 for female for human)] with stromal mouse fibroblasts (negative control; pooled) with the plates for incubations, application medium and maintenance medium are acquired.

Metabolism by suspended hepatocytes. Primary pooled cryopreserved hepatocytes are reconstituted in pre-warmed William's E media containing 10% FCS, 0.05 mg/mL streptomycin and 50 U/mL penicillin and 0.4 mM L-glutamine; and 0.01 mg/mL gentamicin, 0.048 mg/mL hydrocortisone and 0.004 mg/mL insulin, to a final suspension density of 1×106 cells/mL. The incubation was performed fully automatically with Liquid Handling System (Tecan) equipped with a CO2 incubator with an orbital shaker. After the addition of a test compound at e.g. 1 μM to the wells (1×105 cells/well), the 96-well hepatocyte suspension culture plates are incubated in a 5% CO2 at 37 °C. Samples are quenched by addition of acetonitrile (including an internal standard) to the incubation well at the designated time points up to 2 h.

Metabolism by HepatoPac®. Incubations for a test article (at e.g. 1 μM, 0.1% v/v DMSO) as conducted in suspension assays are performed in 96-well plates containing either a co-culture of adherent hepatocytes with mouse fibroblast control cells or control cells alone (5% CO2 atmosphere and 37 °C). The incubation media in human HepatoPac® is identical with that in suspended hepatocytes. At defined time points (2, 18, 26, 48, 72 and 96 h), whole wells are quenched with ice-cold acetonitrile containing an internal standard. Samples are then centrifuged appropriately and the supernatant analyzed by LC-MS/MS. The incubation is conducted in n=1 or 2.

hERG Screening Assay

In the drug development process of small molecules, one of the most frequent adverse side effects, leading to the failure of drugs, is the cardiac arrhythmias. Such failure is often related to the capacity of the drug to inhibit the human ether-i-go-go-related gene (hERG) cardiac potassium channel. Having no or low inhibition of the hERG cardiac potassium channel is therefore considered as beneficial.

### Cells

The CHO crelox hERG cell line (ATCC reference Nr. PTA-6812, female Chinese hamster cells) was generated and validated at Roche. Ready-to-use frozen instant CHO-hERG cells were cryopreserved at Evotec (Germany) and used directly in the experiments.

### Experimental Solutions

The extracellular solution contains (in mM): NaCl 150; KCl 4; CaCl₂ 1; MgCl₂ 1; HEPES 10; pH 7.2-7.4 with NaOH, osmolarity 290-330 mOsm. The internal solution contains (in mM): KCl, 10; KF, 100; NaCl, 10; HEPES, 10; EGTA, 20; pH=7.0-7.4 with KOH, osmolarity 260-300 mOsm.

### Electrophysiology

The effects of a compound on hERG K+-currents parameters will be evaluated at 2 concentrations in at least 4 cells.

The hERG test is performed using automated patch clamp system SynchroPatch® 384 (Nanion Technologies GmbH, Germany). K+ currents are measured with the patch-voltage-clamp technique in the whole-cell configuration at 35-37 °C.

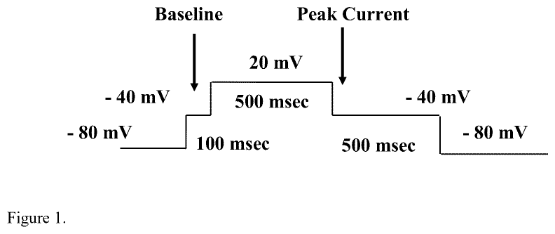

Cells were held at a resting voltage of −80 mV and they were stimulated by a voltage pattern shown in FIG. 1 (pulse pattern used to elicit outward K+ current at 35-37 °C) to activate hERG channels and conduct outward IKhERG current, at a stimulation frequency of 0.1 Hz (6 bpm)

### Data Analysis

The amplitudes of IKhERG were recorded in each concentration of drug and they were compared to the vehicle control values (taken as 100%) to define fractional blocks. The concentration-response data were fitted with the following relationship:

| | |
|---|---|
| | $I ( C ) = \frac{100}{1 + {( C / IC 50 )}^{h}}$ |
| where | C is the concentration, |
| | IC₅₀ is the concentration producing 50% block |
| | h is the Hill coefficient. |

Concentration-response curves were fitted by non-linear regression analysis using EworkBook suite (ID Business Solutions Ltd, UK). Data fit was done with the 4 Parameter Logistic Model (fit=(A+(B/(1+((x/C)\hat{ }D)))), where A=0 and B=100).

The results of the hERG assay are summarised in Table 2 below as hERG IC₂₀.

### Transcellular P-gp Assay:

The general assay uses transfected LLC-PK1 cells (porcine kidney epithelial cells) over-expressing human or mouse P-gp, cultured on 96 well semi-permeable filter membrane plates, where they form a polarized monolayer with tight junctions, and act as a barrier between the apical and basolateral compartment.

P-gp is expressed in the apical-facing membrane of the monolayer.

The tightness of the cell monolayer and functional activity of P-gp are confirmed by addition of a cell-impermeable marker, Lucifer yellow, and a reference P-gp substrate, edoxaban, respectively.

### PAMPA:

PAMPA (Parallel Artificial Membrane Permeability Assay) is a first line permeability screen for drug candidates. The PAMPA assay mimics the transcellular absorption conditions using an artificial phospholipid membrane. This assay determines a permeability value that can be used for compound optimization and ranking purposes as well as input parameters for in silico models to predict intestinal absorption.

The donor concentration is measured at t-start (reference) and compared with the donor and acceptor concentration after a certain time (t-end) to calculate the extent of passage of the compound through the membrane.

### Bacterial Reverse Mutation Test (AMES):

The testing of compounds is conducted as outlined in this guideline: Test No. 471: Bacterial Reverse Mutation Test|OECD Guidelines for the Testing of Chemicals, Section 4: Health Effects|OECD iLibrary (oecd-ilibrary.org)

### Bacteria Culture:

The bacterial strains used are TA98, TA100, TA1535, TA97a and TA102. Batches of each strain, are maintained as frozen stocks. Vials are thawed and used to inoculate cultures in nutrient broth. The cultures are placed in an incubator set to 37 °C with agitation for approximately 10 hours to provide a working culture of at least 108 cells per mL.

To ensure cultures are at the appropriate phase of growth and culture density, a sample is taken from each culture at the end of the incubation period and assessed for culture density by either viability plating or OD650 assessment.

### Treatment:

3 replicates per concentration of compound and positive controls and 6 replicates per vehicle controls are included.

Formulations are prepared using DMSO to allow maximum exposure up to the solubility limit or 1000 g/well for a freely soluble test article. This concentration is equivalent to 5000 g/plate as used in the usual plate incorporation Ames assay.

Concentrations are usually separated by half-log intervals in a single experiment. For soluble compounds, concentrations will be 0, 3.2, 10, 32, 100, 320, 1000 g/well.

### Positive Controls Used are:

### ABBREVIATION NAME USED FOR STRAIN

2NF 2-Nitrofluorene TA98-S-9
NaN3 Sodium Azide TA100 and TA1535-S-9
AAC 9-Aminoacridine TA97a-S-9
MMC Mitomycin C TA102-S-9
B[a]P Benzo[a]pyrene TA98+S-9
AAN Aminoanthracene TA100, TA1535,TA97a and TA102+S-9

Platings will be achieved by the following sequence of additions to 400 μL supplemented molten agar at 45±1 °C:
20 μL of bacterial culture
20 μL of test article solution/vehicle control/positive control
100 μL of 10% S-9 mix or buffer solution
followed by rapid mixing and pouring onto mutation plates (wells).

When set, the plates will be inverted and incubated protected from light for 2 to 3 days in an incubator set to 37 °C.

### Toxicity:

Toxicity is detected by the following parameters:
Diminution of background lawn
Marked reduction in revertants compared to the concurrent vehicle controls
Reduction in mutagenic response.

### Scoring:

Scoring of bacteria colonies is performed manually or electronically using automated colony counter.

### In Vitro Mammalian Cell Micronucleus Test:

The testing of compounds is conducted as outlined in this guideline: Test No. 487: In Vitro Mammalian Cell Micronucleus Test|OECD Guidelines for the Testing of Chemicals, Section 4: Health Effects|OECD iLibrary (oecd-ilibrary.org)

### Cell Culture:

Cultures are maintained in tissue culture flasks containing HEPES-buffered RPMI 1640 medium with GlutaMAX-1 including 10% (v/v) heat inactivated foetal calf serum, 100 Units/mL/100 μg/mL penicillin/streptomycin in a humidified incubator set to 37 °C, 5% (v/v) CO2 in air. Cells will be subcultured at low to medium density at least once prior to treatment. On the day prior to treatment, cells will be subcultured at a density of approximately 7×104 cells/mL. Cells will be maintained at 37 °C, 5% (v/v) CO2 in air, in a humidified environment prior to treatment.

### Treatment:

Cultured human lymphoblastoid TK6 cells will be exposed to the compound for 3 hours in the presence of S-9, followed by a recovery period of 24 hours. In addition, a continuous 27 hour treatment in the absence of S-9 will be included as a number of chemicals have been reported as only exerting positive effects following prolonged treatment. This is equivalent to approximately 1.5-2.0 times the average generation time of the TK6 cells used in this laboratory (cell cycle approximately 15 hours). All cultures will be sampled 27 hours after the beginning of treatment.

Dilutions will be prepared in DMSO that allow maximum exposure up to the solubility limit, 1 mM or 500 μg/mL, whichever is lower.

Normally, at least 12 concentrations separated by 0.7-fold intervals, ranging down from the upper limit (for soluble compounds with a MW≥500, concentrations will be 9.887, 14.12, 20.18, 28.82, 41.18, 58.82, 84.04, 120.1, 171.5, 245, 350 and 500 μg/mL). The final concentration of DMSO will be 1% v/v. Positive controls are Noscapine in the absence of S-9 and Cyclophosphamide in the presence of S-9. 2 replicates per concentration of compound and multiple concurrent vehicle and positive controls will be included per treatment in 96-well plates and incubated for the treatment time at 37 °C, 5% (v/v) CO₂. 3 hour treatment cultures will be washed once and reincubated with fresh medium for 24 hours.

### Harvesting:

At the defined sampling time an aliquot of cell suspension from designated cultures will be taken for determination of cell number by using a Coulter Counter. Cultures designated for analysis will be centrifuged at approximately 200 g, 5 minutes. Cells will be resuspended in 0.075 M KCl prior to fixation in fresh, cold methanol/glacial acetic acid (7:1 v/v). Fixed cells will be stored in fixative at 2-8 °C prior to slide preparation.

Slides will be air-dried prior to staining by immersion in 12.5 μg/mL Acridine Orange in phosphate buffered saline (PBS), pH 6.8 for approximately 10 minutes, following by a wash with PBS (with agitation) for a few seconds.

### Cytotoxicity Readout and Selection of Concentrations:

Toxicity is expressed as Population Doubling (PD) relative to vehicle controls. PD will be calculated for each concentration as follows:

PD=[log(N/X₀)]/log 2

Where N=mean final cell count/culture at each concentration
X₀=starting (baseline) count

The highest concentration for micronucleus analysis should either not exceed (approximately) 50% cytotoxicity, be the highest concentration tested, or, be the lowest precipitating concentration observed by eye at the end of the treatment incubation period. Slides from the highest selected concentration and at least two lower concentrations will be analysed, such that a range of cytotoxicity from maximum to little or none is covered, where appropriate. A minimum of 1000 mononucleate cells from each culture (2000 per concentration) will be analysed for micronuclei.

### Evaluation Criteria:

The compound will be considered to induce clastogenic and/or aneugenic events if:
A statistically significant increase in the frequency of MNMON cells at one or more concentrations is observed.
The incidence of cells with micronuclei at such a concentration exceeds the normal range in both replicates.
A concentration-related increase in the proportion of cells with micronuclei is observed (positive trend test).

The compound will be considered positive in this assay if all of the above criteria are met.

The compound will be considered negative in this assay if none of the above criteria are met.

Results which only partially satisfy the above criteria will be dealt with on a case-by-case basis, but in the context of the screening study, will be concluded as either positive, negative or equivocal. Evidence of a concentration-related effect is considered useful but not essential in the evaluation of a positive result. Biological relevance will be taken into account, for example consistency of response within and between concentrations and (if applicable) between experiments, or effects occurring only at very toxic concentrations.

TABLE 1
NLRP3 inhibitory activity

| | THP-1 | Human |
|---|---|---|
| | pyroptosis | whole blood |
| Example | Assay | IL-1β Assay |
| No. | IC₅₀ (nM) | IC₅₀ (nM) |
| 1 | 5 | 54 |
| 1A | 3 | 43 |
| 1B | 143 | |
| 2 | 6 | 40 |
| 2A | 304 | |
| 2B | 4 | 19 |
| 3 | 99 | 108 |
| 4A | 134 | |
| 4B | 211 | |
| 5 | 38 | 37 |
| 5A | 29 | 32 |
| 5B | 64 | 36 |
| 6A | 58 | 47 |
| 6B | 63 | 59 |
| 7 | 3.0 | 36 |
| 8 | 21 | |
| 9 | 56 | 43 |
| 10 | 11 | 75 |
| 11 | 57 | 51 |
| 13 | 10 | 41 |
| 14a | 7 | 12 |
| 14b | 171 | |
| 15 | 25 | 23 |

TABLE 2
hERG inhibition

| Example No | IC₂₀ [μM] |
|---|---|
| 2B | >10 |
| 15 | >10 |

The invention will now be illustrated by the following examples which have no limiting character.

In case the preparative examples are obtained as a mixture of enantiomers or diastereoisomers, the pure enantiomers or diastereomers can be obtained by methods described herein or by methods known to those skilled in the art, such as e.g. chiral chromatography or crystallization.

### EXPERIMENTAL METHODS

Abbreviations

| | |
|---|---|
| aq | aqueous |
| ESI | Electrospray ionization |
| h(s) | hour(s) |
| DCM | Dichloromethane |
| DEA | Diethylamine |
| DMSO | dimethylsulfoxide |
| HATU | Hexafluorophosphate Azabenzotriazole Tetramethyl Uronium |
| HPLC | High-performance liquid chromatography |
| LCMS | Liquid chromatography-mass spectrometry |
| MeOH | methanol |
| rac | racemic |
| rel | relative |
| Sat. | saturated |
| SFC | Supercritical fluid chromatography |

### Examples

### Example 1: 5-[4-[[rac-(1R,2R)-2-Hydroxycyclohexyl]amino]pyrido[3,4-d]pyridazin-1-yl]indan-4-ol

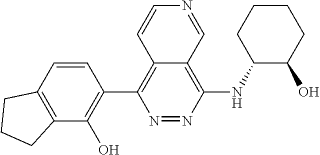 [SMILES: Oc1c(-c2nnc(N[C@@H]3CCCC[C@H]3O)c3cnccc23)ccc2c1CCC2]

### Step A: rac-(1R,2R)-2-[(1-Chloropyrido[3,4-d]pyridazin-4-yl)amino]cyclohexanol

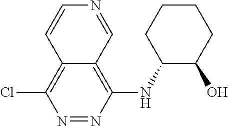 [SMILES: O[C@@H]1CCCC[C@H]1Nc1nnc(Cl)c2ccncc12]

To a mixture of 1,4-dichloropyrido[3,4-d]pyridazine (CAS #14490-19-8, 1.01 g, 4.81 mmol, 1.0 eq) and trans-2-aminocyclohexanol (CAS #6982-39-4, 866 mg, 7.22 mmol, 1.50 eq) in 1,4-dioxane (20 mL) was added N,N-diisopropylethylamine (CAS #7087-68-5, 1.87 g, 2.52 mL, 14.4 mmol, 3.0 eq). The reaction mixture was stirred at 95 °C for 16 h. The reaction was quenched with water (30 mL) and extracted with aq. NaHCO₃ (80 mL) and ethyl acetate (2×80 mL). The organic layers were washed with brine (80 mL), dried over sodium sulfate, filtered off and concentrated in vacuo. The crude product was purified by flash chromatography (silica gel, 25 g, gradient 0% to 100% ethyl acetate in heptane) to afford the title compound (210 mg, 15% yield) as a yellow solid. LCMS: m/z 279.1 ([{35Cl}M+H]⁺), 281.0 ([{37Cl}M+H]⁺), ESI pos.

### Step B: 5-[4-[[rac-(1R,2R)-2-Hydroxycyclohexyl]amino]pyrido[3,4-d]pyridazin-1-yl]indan-4-ol

In a sealable tube, a mixture of aforementioned rac-(1R,2R)-2-[(1-chloropyrido[3,4-d]pyridazin-4-yl)amino]cyclohexanol (110 mg, 0.375 mmol, 1.0 eq), 5-(4,4,5,5-tetramethyl-1,3,2-dioxaborolan-2-yl)indan-4-ol (CAS #2795101-92-5, 139 mg, 0.524 mmol, 1.4 eq) and cesium carbonate (366 mg, 1.12 mmol, 3.0 eq) in 1,4-dioxane (3.6 mL) and water (0.9 mL) was set under argon, and XPhos Pd G3 gt (CAS #1445085-55-1, 48 mg, 0.056 mmol, 0.15 eq) was added. The reaction mixture was stirred at 100 °C in the sealed tube for 3 h. The reaction mixture was cooled to room temperature, quenched with water (20 mL) and saturated NH₄Cl solution (20 mL), then extracted with ethyl acetate (2×40 mL). The organic layers were washed with brine (40 mL), dried over sodium sulfate, filtered off and concentrated in vacuo. The crude product was purified by flash chromatography (silica gel, 25 g, gradient 0% to 50% (dichloromethane:methanol:NH₄OH 110:10:1) in dichloromethane) to afford the title compound (53 mg, 36% yield) as a yellow foam. LCMS: m/z 377.2 [M+H]⁺, ESI pos.

### Examples 1A and 1B

### 5-[4-[[rel-(1R,2R)-2-hydroxycyclohexyl]amino]pyrido[3,4-d]pyridazin-1-yl]indan-4-ol (1A) and 5-[4-[[rel-(1S,2S)-2-hydroxycyclohexyl]amino]pyrido[3,4-d]pyridazin-1-yl]indan-4-ol (1B)

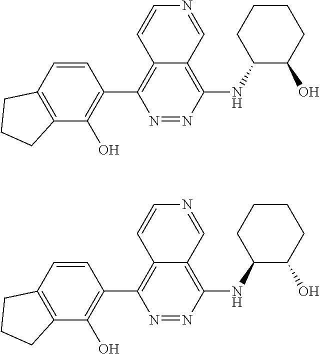 [SMILES: Oc1c(-c2nnc(N[C@@H]3CCCC[C@H]3O)c3cnccc23)ccc2c1CCC2 ; Oc1c(-c2nnc(N[C@H]3CCCC[C@@H]3O)c3cnccc23)ccc2c1CCC2]

Chiral separation of the aforementioned compound 1 was done by SFC (Column chiral OZ-H, 5 μm, 250×20 mm; elution: 40% methanol in CO₂) to yield the enantiopure, first eluting (retention time=1.79 min) title compound 1A (16 mg, 40% yield) as a yellow foam (LCMS: m/z 377.2 [M+H]⁺, ESI pos.) and the second eluting (retention time=2.64 min), enantiopure title compound 1B (15 mg, 38% yield) as a yellow foam.

### Example 2

### 5-[4-[[rac-(1R,2R)-2-Hydroxycyclohexyl]amino]pyrido[3,4-d]pyridazin-1-yl]-2,3-dihydrobenzofuran-4-ol

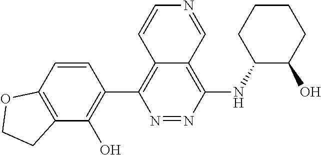 [SMILES: Oc1c(-c2nnc(N[C@@H]3CCCC[C@H]3O)c3cnccc23)ccc2c1CCO2]

In a sealable tube, a mixture of aforementioned rac-(1R,2R)-2-[(1-chloropyrido[3,4-d]pyridazin-4-yl)amino]cyclohexanol (example 1, step A) (148 mg, 0.50 mmol, 1.0 eq), 5-(4,4,5,5-tetramethyl-1,3,2-dioxaborolan-2-yl)coumaran-4-ol (CAS #2923540-32-1, 195 mg, 0.71 mmol, 1.40 eq) and cesium carbonate (493 mg, 1.51 mmol, 3.00 eq) in 1,4-dioxane (4 mL) and water (1 mL) was set under argon, and XPhos Pd G3 gt (CAS #1445085-55-1, 64 mg, 0.076 mmol, 0.150 eq) was added. The reaction mixture was stirred at 100 °C in the sealed tube for 5 h. The reaction mixture was cooled to room temperature, quenched with water (20 mL) and saturated NH₄Cl solution (20 mL), then extracted with ethyl acetate (2×40 mL). The organic layers were washed with brine (40 mL), dried over sodium sulfate, filtered off and concentrated in vacuo. The crude product was purified by flash chromatography (silica gel, 40 g, gradient 0% to 50% (dichloromethane:methanol:NH₄OH 110:10:1) in dichloromethane) to afford the title compound (55 mg, 27% yield) as a yellow foam. LCMS: m/z 379.1 [M+H]⁺, ESI pos.

### Examples 2A and 2B

### 5-[4-[[rel-(1R,2R)-2-Hydroxycyclohexyl]amino]pyrido[3,4-d]pyridazin-1-yl]-2,3-dihydrobenzofuran-4-ol (2A) 5-[4-[[rel-(1S,2S)-2-Hydroxycyclohexyl]amino]pyrido[3,4-d]pyridazin-1-yl]-2,3-dihydrobenzofuran-4-ol (2B)

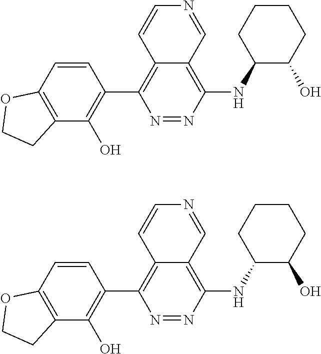 [SMILES: Oc1c(-c2nnc(N[C@H]3CCCC[C@@H]3O)c3cnccc23)ccc2c1CCO2 ; Oc1c(-c2nnc(N[C@@H]3CCCC[C@H]3O)c3cnccc23)ccc2c1CCO2]

Chiral separation of the aforementioned compound 2 was done by SFC (Column chiral OJ-H, 5 μm, 250×20 mm; elution: 30% methanol+0.2% diethylamine in CO₂) to yield the enantiopure, first eluting (retention time=2.52 min) title compound 2A (19 mg, 45% yield) as a light brown foam (LCMS: m/z 379.1 [M+H]⁺, ESI pos) and the second eluting (retention time=3.53 min) enantiopure title compound 2B (18 mg, 43% yield) as a light brown foam. LCMS: m/z 379.1 [M+H]⁺, ESI pos.

### Example 3

### rac-(1R,2R)-2-[[1-(1H-Indol-6-yl)pyrido[3,4-d]pyridazin-4-yl]amino]cyclohexanol

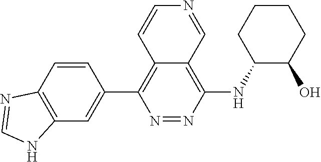 [SMILES: O[C@@H]1CCCC[C@H]1Nc1nnc(-c2ccc3nc[nH]c3c2)c2ccncc12]

In a sealable tube, a mixture of aforementioned rac-(1R,2R)-2-[(1-chloropyrido[3,4-d]pyridazin-4-yl)amino]cyclohexanol (example 1, step A) (180 mg, 0.6 mmol, 1.00 eq), 1H-indol-6-ylboronic acid (CAS #147621-18-9, 138 mg, 0.86 mmol, 1.40 eq) and cesium carbonate (600.0 mg, 1.84 mmol, 3.0 eq) in 1,4-dioxane (6 mL) and water (1.5 mL) was set under argon, and XPhos Pd G3 gt (CAS #1445085-55-1, 78 mg, 0.092 mmol, 0.150 eq) was added. The reaction mixture was stirred in the sealed tube for 3 h at 100 °C. The reaction mixture was cooled to room temperature, quenched with water (25 mL) and saturated NH₄Cl solution (20 mL), then extracted with ethyl acetate (2×40 mL). The organic layers were washed with brine (40 mL), dried over sodium sulfate, filtered off and concentrated in vacuo. The crude product was purified by flash chromatography (silica gel, 25 g, gradient 0% to 50% (dichloromethane:methanol:NH₄OH 110:10:1) in dichloromethane) to afford the title compound (15 mg, 6% yield) as a yellow solid. LCMS: m/z 360.2 [M+H]⁺, ESI pos.

### Example 4A and 4B

### 1-(3-Fluoro-1H-indol-6-yl)-N-[[rel-(3R)-tetrahydrofuran-3-yl]methyl]pyrido[3,4-d]pyridazin-4-amine (4A) and 1-(3-fluoro-1H-indol-6-yl)-N-[[rel-(3S)-tetrahydrofuran-3-yl]methyl]pyrido[3,4-d]pyridazin-4-amine (4B)

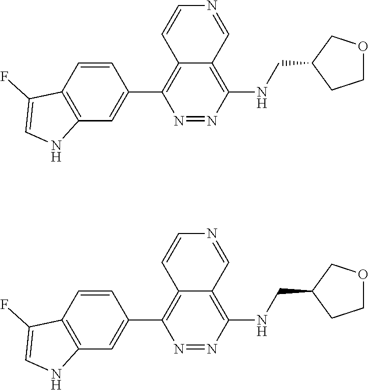 [SMILES: Fc1c[nH]c2cc(-c3nnc(NC[C@H]4CCOC4)c4cnccc34)ccc12 ; Fc1c[nH]c2cc(-c3nnc(NC[C@@H]4CCOC4)c4cnccc34)ccc12]

### Step A: 1-Chloro-N-(tetrahydrofuran-3-ylmethyl)pyrido[3,4-d]pyridazin-4-amine

To a mixture of 1,4-dichloropyrido[3,4-d]pyridazine (CAS #14490-19-8, 333 mg, 1.58 mmol, 1.000 eq) and (tetrahydrofuran-3-yl)methanamine (CAS #165253-31-6, 242 mg, 2.39 mmol, 1.51 eq) in 1,4-dioxane (3 mL) was added N,N-diisopropylethylamine (618 mg, 0.83 mL, 4.78 mmol, 3.02 eq). The reaction mixture was stirred at 90 °C for 16 h. The reaction was quenched with water (10 mL) and extracted with satured NaHCO₃ solution (20 mL) and ethyl acetate (2×40 mL). The organic layers were washed with brine (20 mL), dried over sodium sulfate, filtered off and concentrated in vacuo. The crude product was purified by flash chromatography (silica gel, 25 g, gradient 0% to 100% ethyl acetate in heptane) to afford a mixture containing the title compound and the corresponding regioisomer (466 mg, 88% yield, ratio determined using ¹H-NMR: 1:0.42) as a light yellow solid. LCMS: m/z 265.1 ([{³⁵Cl}M+H]⁺), 267.1 ([{³⁷Cl}M+H]⁺), ESI pos.

### Step B: 1-(3-Fluoro-1H-indol-6-yl)-N-[[rel-(3R)-tetrahydrofuran-3-yl]methyl]pyrido[3,4-d]pyridazin-4-amine (4A) and 1-(3-fluoro-1H-indol-6-yl)-N-[[rel-(3S)-tetrahydrofuran-3-yl]methyl]pyrido[3,4-d]pyridazin-4-amine (4B)

The aforementioned mixture (step A) (84 mg, 0.32 mmol, 1.00 eq), 3-fluoro-6-(4,4,5,5-tetramethyl-1,3,2-dioxaborolan-2-yl)-1H-indole (116 mg, 0.44 mmol, 1.40 eq) and potassium carbonate (CAS #584-08-7, 211 mg, 1.52 mmol, 4.80 eq) was dissolved in 1,4-dioxane (2 mL) and water (1 mL) under argon. Then dichloro[1,1′-bis(diphenylphosphino)ferrocene]palladium(ii)dichloromethane adduct (CAS #95464-05-4, 39 mg, 0.048 mmol, 0.150 eq) was added. The reaction mixture was stirred in a sealed tube at 95 °C for 6 h. The reaction mixture was cooled to room temperature and extracted with ethyl acetate (30 mL) and water (30 mL). The aqueous layer was backextracted with ethyl acetate (30 mL). The organic layers were washed with water (20 mL) and brine (20 mL). The combined organic layers were dried over sodium sulfate, filtered and concentrated in vacuo. The crude product was purified by flash chromatography (silica gel, 12 g, gradient 0% to 50% (dichloromethane:methanol:NH₄OH 110:10:1) in dichloromethane) to afford the title compound (67 mg, 55% yield) as a light yellow solid (containing only one regiosisomer). Chiral separation of the title compound was done by SFC (Column chiral OJ-H, 5 μm, 250×20 mm; elution: 30% methanol+0.2% diethylamine in CO₂) to yield the enantiopure, first eluting (retention time=2.95 min) title compound 4A (21 mg, 31% yield) as a light yellow solid (LCMS: m/z 364.2 [M+H]⁺, ESI pos) and the second eluting (retention time=3.27 min), enantiopure title compound 4B (12 mg, 18% yield) as a light yellow solid. LCMS: m/z 364.2 [M+H]⁺, ESI pos.

### Example 5

### 5-[4-[[rac-(3S)-Tetrahydrofuran-3-yl]methylamino]pyrido[3,4-d]pyridazin-1-yl]-2,3-dihydrobenzofuran-4-ol

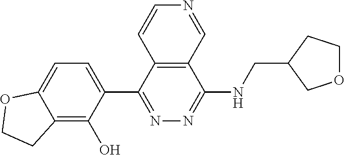 [SMILES: Oc1c(-c2nnc(NCC3CCOC3)c3cnccc23)ccc2c1CCO2]

A mixture of 5-(4,4,5,5-tetramethyl-1,3,2-dioxaborolan-2-yl)coumaran-4-ol (CAS #2923540-32-1, 91 mg, 312 μmol, 1.5 eq), aforementioned mixture (Example 4, step A) (110 mg, 208 μmol, 1.0 eq) and cesium carbonate (203 mg, 623 μmol, 3.0 eq) in water (0.32 mL) and 1,4-dioxane, extra dry (1.28 mL) was flushed with argon. Then XPhos Pd G3 (26.4 mg, 31.2 μmol, 0.15 eq) was added and the mixture was stirred at 95 °C for 2 h. The reaction mixture was cooled to room temperature and extracted with ethyl acetate and water. The aqueous layer was back-extracted with ethyl acetate two times. The organic layers were washed with water and brine. The combined organic layers were dried over sodium sulfate, filtered and concentrated in vacuo. The crude product was first purified by flash chromatography on silica gel (gradient 0% to 10% methanol in dichloromethane) and then via SFC (achiral 100PEI, 12 n, 5 μm, 250×20 mm; 18% MeOH) to afford the title compound (12 mg, 13%) as a yellow solid and its region-isomer (24 mg, 30%). LCMS m/z: 365.3 [M+H]⁺, ESI pos.

### Examples 5A and 5B

### 5-[4-[[rel-(3R)-tetrahydrofuran-3-yl]methylamino]pyrido[3,4-d]pyridazin-1-yl]-2,3-dihydrobenzofuran-4-ol (5A) and 5-[4-[[rel-(3S)-tetrahydrofuran-3-yl]methylamino]pyrido[3,4-d]pyridazin-1-yl]-2,3-dihydrobenzofuran-4-ol (5B)

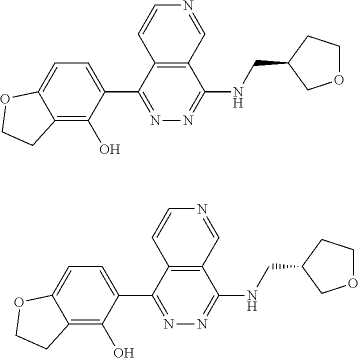 [SMILES: Oc1c(-c2nnc(NC[C@H]3CCOC3)c3cnccc23)ccc2c1CCO2 ; Oc1c(-c2nnc(NC[C@@H]3CCOC3)c3cnccc23)ccc2c1CCO2]

Chiral separation of the aforementioned compound 5 was done by SFC (Column chiral IJ, 5 μm, 250×20 mm; elution: 27% MeOH+0.2% DEA) to yield the enantiopure, first eluting (retention time=2.74 min) title compound 5A (9 mg, 45% yield) (LCMS: m/z 365.3 [M+H]⁺, ESI pos) and the second eluting (retention time=3.07 min) enantiopure title compound 5B (8 mg, 40% yield) both as yellow solids. LCMS m/z 365.3 [M+H]⁺, ESI pos.

### Example 6A and 6B

### 5-[4-[[rel-(2R)-Tetrahydrofuran-2-yl]methylamino]pyrido[3,4-d]pyridazin-1-yl]-2,3-dihydrobenzofuran-4-ol (6A) and 5-[4-[[rel-(2S)-Tetrahydrofuran-2-yl]methylamino]pyrido[3,4-d]pyridazin-1-yl]-2,3-dihydrobenzofuran-4-ol (6B)

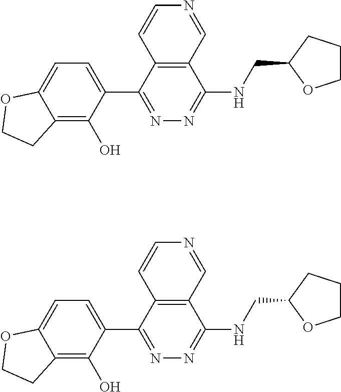 [SMILES: Oc1c(-c2nnc(NC[C@H]3CCCO3)c3cnccc23)ccc2c1CCO2 ; Oc1c(-c2nnc(NC[C@@H]3CCCO3)c3cnccc23)ccc2c1CCO2]

### Step A: 1-Chloro-N-(tetrahydrofuran-2-ylmethyl)pyrido[3,4-d]pyridazin-4-amine

To a mixture of commercially available 1,4-dichloropyrido[3,4-d]pyridazine (CAS #14490-19-8200 mg, 950 μmol, 1.0 eq) and tetrahydrofurfurylamine (CAS #4795-29-3, 145 mg, 148 μL, 1.44 mmol, 1.5 eq) in 1,4-dioxane (1 mL) was added NN-diisopropylethylamine (371 mg, 0.50 mL, 2.87 mmol, 3.0 eq). The reaction mixture was stirred at 100 °C for 16 h. The reaction mixture was extracted with ethyl acetate (~50 mL) and water (~10 mL). The aqueous layer was back-extracted with ethyl acetate (~50 mL). The organic layers were washed with water (~10 mL) and brine (~5 mL). The combined organic layers were dried over sodium sulfate, filtered and concentrated in vacuo. The residue was purified by flash chromatography on silica gel (gradient 0% to 10% MeOH in EtOAc) to afford the title compound and its regioisomer (196 mg, 38%; ratio determined using ¹H-NMR: 1:0.48) as light yellow solid. LCMS m/z: 265.1 [M+H]⁺, ESI pos.

### Step B: 5-[4-[[rel-(2R)-Tetrahydrofuran-2-yl]methylamino]pyrido[3,4-d]pyridazin-1-yl]-2,3-dihydrobenzofuran-4-ol and 5-[4-[[rel-(2S)-Tetrahydrofuran-2-yl]methylamino]pyrido[3,4-d]pyridazin-1-yl]-2,3-dihydrobenzofuran-4-ol

A mixture of 5-(4,4,5,5-tetramethyl-1,3,2-dioxaborolan-2-yl)coumaran-4-ol (80.9 mg, 278 μmol, 1.5 eq), XPhos Pd G3 (23.5 mg, 27.8 μmol, 0.15 eq), aforementioned mixture (Example 6, step A) (100 mg, 185 μmol, 1.0 eq) and cesium carbonate (181 mg, 555 μmol, 3.0 eq) in water (0.28 mL) and 1,4-dioxane, extra dry (1.14 mL) was flushed with argon and stirred at 95 °C for 2 h. The reaction mixture was cooled to room temperature and extracted with ethyl acetate and water. The aqueous layer was back-extracted with ethyl acetate two times. The organic layers were washed with water and brine. The combined organic layers were dried over sodium sulfate, filtered and concentrated in vacuo. The crude product was first purified by flash chromatography on silica gel (gradient 0% to 10% methanol in dichloromethane), then by SFC (Column chiral OJ-H, 5 μm, 250×20 mm; elution: 25% MeOH+0.2% DEA) to yield the racemate, first eluting (retention time=1.89 min) regioisomer (15 mg, 21%) as a yellow solid (LCMS m/z: 365.3 [M+H]⁺, ESI pos), the enantiopure second eluting (retention time=2.72 min) title compound 6A (15 mg, 21% yield) as a brown solid (LCMS m/z: 365.3[M+H]⁺, ESI pos) and the enantiopure third eluting (retention time=3.13 min) title compound 6B (20 mg, 28% yield) as a brown solid (LCMS m/z: 365.3[M+H]⁺, ESI pos).

### Example 7

### 3-[4-[[rac-(JR,2R)-2-Hydroxycyclohexyl]amino]pyrido[3,4-d]pyridazin-1-yl]bicyclo[4.2.0]octa-1,3,5-trien-2-ol

### Step A: 3-(4,4,5,5-Tetramethyl-1,3,2-dioxaborolan-2-yl)bicyclo[4.2.0]octa-1(6),2,4-trien-2-ol

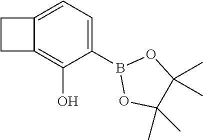 [SMILES: CC1(C)OB(c2ccc3c(c2O)CC3)OC1(C)C]

A mixture of 3-bromobicyclo[4.2.0]octa-1(6),2,4-trien-2-ol (317 mg, 1.59 mmol, 1.0 eq), bis(pinacolato)diboron (2028.8 mg, 7.99 mmol, 5.02 eq), Pd(dppf)Cl₂·DCM Complex (158.5 mg, 0.19 mmol, 0.12 eq), and potassium acetate (475.5 mg, 4.85 mmol, 3.04 eq) in 1,4-Dioxane (15 mL) was degassed with N₂ for 5 mins, then was heated to 90 °C and stirred for 2 h. The reaction was allowed to cool to rt, then was concentrated in vacuo and purified by column chromatography on silica gel (40 g cartridge, 0-100% EtOAc/isohexane) to give the title compound (144.2 mg, 14% yield) as a white solid. A further batch of the title compound (366 mg, 25% yield) was also isolated as a white solid. ¹H NMR (500 MHz, DMSO) δ 8.55 (s, 1H), 7.36 (d, 1H), 6.58 (d, 1H), 3.12-2.97 (m, 4H), 1.28 (s, 12H). LCMS m/z 247.7 [M+H]⁺ ESI pos.

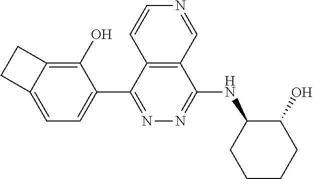 [SMILES: Oc1c(-c2nnc(N[C@@H]3CCCC[C@H]3O)c3cnccc23)ccc2c1CC2]

### Step B: 3-[4-[[rac-(1R,2R)-2-Hydroxycyclohexyl]amino]pyrido[3,4-d]pyridazin-1-yl]bicyclo[4.2.0]octa-1,3,5-trien-2-ol

A mixture of 3-(4,4,5,5-tetramethyl-1,3,2-dioxaborolan-2-yl)bicyclo[4.2.0]octa-1(6),2,4-trien-2-ol (17.0 mg, 0.07 mmol, 1.0 eq), rac-(1R,2R)-2-[(1-chloropyrido[3,4-d]pyridazin-4-yl)amino]cyclohexanol (37.8 mg, 0.08 mmol, 1.1 eq, Example 1, Step A), XPhos Pd G3 (5.9 mg, 0.01 mmol, 0.1 eq) and potassium carbonate (28.6 mg, 0.21 mmol, 3.0 eq) in acetonitrile (2 mL) and water (0.5 mL) was degassed with N₂ for 5 mins, then was heated to 80 °C for ~16 h. The reaction mixture was allowed to cool to rt, then was diluted with water (20 mL) and EtOAc (20 mL). The layers were separated and the aqueous layer was washed with EtOAc (2×20 mL). The combined organic layers were washed with brine (20 mL), dried with MgSO₄ and concentrated in vacuo. The crude product was purified by column chromatography on silica gel (40 g, 0-5% MeOH(NH₃)/DCM) then re-purified by RP column chromatography (40 g, 0.1% ammonium hydroxide, 5-40% MeCN/water) to afford the title compound (4.2 mg, 17% yield) as a yellow solid. LCMS m/z 363.3 [M+H]⁺, ESI pos.

### Example 8

### 5-[4-[[(1R,2R)-2-Hydroxycyclohexyl]amino]pyrido[3,4-d]pyridazin-1-yl]-6-(methoxymethyl)-2,3-dihydrobenzofuran-4-ol

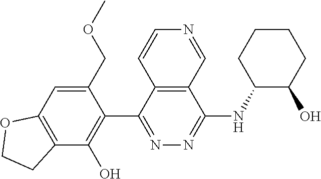 [SMILES: COCc1cc2c(c(O)c1-c1nnc(N[C@@H]3CCCC[C@H]3O)c3cnccc13)CCO2]

### Step A: 2-[2,6-Dibromo-4-(methoxymethyl)phenyl]ethanol

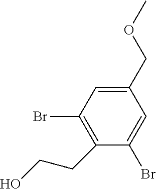

To a solution of 1,3-dibromo-5-(methoxymethyl)benzene (171 g, 610 mmol, 1.00 eq, CAS #1646314-10-4) in THF (750 mL) was added LDA (2.00 M, 366 mL, 1.20 eq) at −78 °C and stirring was continued for 30 mins. Afterwards, a solution of 1,3,2-dioxathiolane 2,2-dioxide (98.5 g, 794 mmol, 1.30 eq, CAS #1072-53-3) in THF (750 mL) was added to the mixture at −78 °C. The mixture was warmed and stirred at 25 °C for 1 h. The reaction mixture was quenched by addition of aq. HCl (12.0 M, 170 mL) at 0 °C, and then diluted with water (500 mL) and extracted with EtOAc (500 mL×3). The combined organic layers were washed with brine (500 mL), dried over Na₂SO₄, filtered and concentrated under reduced pressure. The crude was purified by column chromatography on silica gel (petroleum ether/ethyl acetate=50/1 to 10/1) to yield the title compound (60.0 g, 30% yield) as a white solid.

### Step B: 4-Bromo-6-(methoxymethyl)-2,3-dihydrobenzofuran

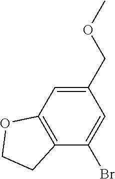

To a solution of 2-[2,6-dibromo-4-(methoxymethyl)phenyl]ethanol (Example 8, step A) (60.0 g, 185 mmol, 1.00 eq) in dioxane (600 mL) was added t-BuOLi (44.4 g, 555 mmol, 50.0 mL, 3.00 eq) and CuI (3.53 g, 18.5 mmol, 0.10 eq) under N₂ atmosphere. The mixture was stirred at 100 °C for 5 h. The reaction mixture was quenched by addition water (1.00 L), and then diluted with ethyl acetate (500 mL) and extracted with ethyl acetate (500 mL×3). The combined organic layers were washed with brine (500 mL), dried over Na₂SO₄, filtered and concentrated under reduced pressure. The title compound (50.0 g, crude) was obtained as a yellow oil and used without further purification in the next step.

### Step C: 6-(Methoxymethyl)-2,3-dihydrobenzofuran-4-ol

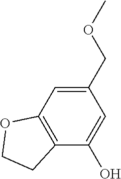

To a solution of 4-bromo-6-(methoxymethyl)-2,3-dihydrobenzofuran (Example 8, step B) (48.0 g, 197 mmol, 1.00 eq) in dioxane (250 mL) and H₂O (250 mL) was added t-BuXphos (16.7 g, 39.4 mmol, 0.20 eq), Pd2 (dba)3 (9.04 g, 9.87 mmol, 0.05 eq) and KOH (33.2 g, 592 mmol, 3.00 eq). The mixture was stirred at 80 °C for 16 h under N₂ atmosphere. The mixture was adjusted to pH=5 with aq. HCl (12 M), and then extracted with ethyl acetate (500 mL×3). The combined organic layers were washed with brine (500 mL), dried over Na₂SO₄, filtered and concentrated under reduced pressure. The crude was purified by column chromatography on silica gel (petroleum ether/ethyl acetate=50/1 to 5/1) to afford the title compound (26 g, 74% yield) as a white solid.

### Step D: 5-Bromo-6-(methoxymethyl)-2,3-dihydrobenzofuran-4-ol

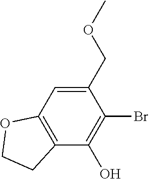

To a solution of 6-(methoxymethyl)-2,3-dihydrobenzofuran-4-ol (Example 8, step C) (25.0 g, 138 mmol, 1.00 eq) in DCM (250 mL) was added NBS (22.2 g, 124 mmol, 0.90 eq) at 0 °C. The mixture was stirred at 0 °C for 5 h. The reaction mixture was concentrated under reduced pressure and the residue was purified by column chromatography on silica gel (petroleum ether/ethyl acetate: 50/1 to 10/1) to yield the title compound (16.0 g, 45% yield) as a white solid. ¹H NMR (400 MHz, CDCl₃) δ [ppm]: 6.56 (s, 1H), 5.72 (s, 1H), 4.63 (t, 2H), 4.44 (s, 2H), 3.43 (s, 3H), 3.22 (t, 2H).

### Step E: 4-Benzyloxy-5-bromo-6-(methoxymethyl)-2,3-dihydrobenzofuran

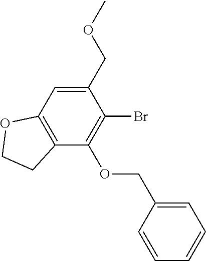 [SMILES: COCc1cc2c(c(OCc3ccccc3)c1Br)CCO2]

To a solution of 5-bromo-6-(methoxymethyl)-2,3-dihydrobenzofuran-4-ol (Example 8, step D) (15.0 g, 57.8 mmol, 1.00 eq) in MeCN (150 mL) was added BnBr (10.8 g, 63.6 mmol, 7.56 mL, 1.10 eq) and K₂CO₃ (16.0 g, 115 mmol, 2.00 eq). The mixture was stirred at 25 °C for 2 h. The reaction mixture was concentrated under reduced pressure to give a residue. The residue was diluted with water (100 mL) and extracted with EtOAc (50 mL×3). The combined organic layers were washed with brine (50 mL), dried over Na₂SO₄, filtered and concentrated under reduced pressure to give a residue. The crude product was triturated with petroleum ether (50.0 mL) at 25 °C for 16 h. The title compound (10.0 g, 74% yield) was obtained as a white solid. LCMS: m/z 350.0 [M+H]⁺, ESI pos.

### Step F: 2-[4-Benzyloxy-6-(methoxymethyl)-2,3-dihydrobenzofuran-5-yl]-4,4,5,5-tetramethyl-1,3,2-dioxaborolane

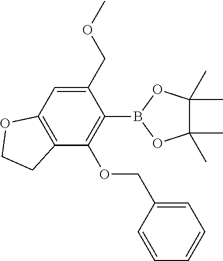 [SMILES: COCc1cc2c(c(OCc3ccccc3)c1B1OC(C)(C)C(C)(C)O1)CCO2]

To a solution of 4-benzyloxy-5-bromo-6-(methoxymethyl)-2,3-dihydrobenzofuran (Example 8, step E) (1.5 g, 4.08 mmol, 1.00 eq) and 2-isopropoxy-4,4,5,5-tetramethyl-1,3,2-dioxaborolane (1.19 g, 1.3 mL, 6.37 mmol, 1.56 eq) in tetrahydrofuran (15 mL) was added dropwise 1.6 M n-butyllithium, solution in hexane (4 mL, 6.4 mmol, 1.57 eq) at −76 °C and stirring was continued at −76 °C for 2.25 h. The reaction mixture was warmed to −60 °C, quenched with saturated aq. NH₄Cl-solution (~10 mL) at −60 °C, warmed to room temperature and then extracted with ethyl acetate (~70 mL) and saturated aq. NH₄Cl-solution (~10 mL). The aqueous layer was backextracted with ethyl acetate (~70 mL). The organic layers were washed with brine (~10 mL). The combined organic layers were dried over sodium sulfate, filtered and concentrated in vacuo. The crude product was purified by flash chromatography (silica gel, 25 g, gradient 0% to 20% ethyl acetate in heptane) to afford the title compound (1.26 g, 74% yield) as colorless oil. LCMS: m/z 397.3 [M+H]⁺, ESI pos.

### Step G: 6-(Methoxymethyl)-5-(4,4,5,5-tetramethyl-1,3,2-dioxaborolan-2-yl)-2,3-dihydrobenzofuran-4-ol

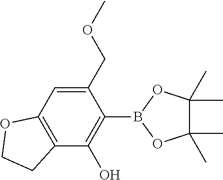 [SMILES: COCc1cc2c(c(O)c1B1OC(C)(C)C(C)(C)O1)CCO2]

A solution of 2-[4-benzyloxy-6-(methoxymethyl)-2,3-dihydrobenzofuran-5-yl]-4,4,5,5-tetramethyl-1,3,2-dioxaborolane (Example 8, step F) (1.24 g, 2.98 mmol, 1.00 eq) in ethyl acetate (15 mL) and methanol (2.5 mL) was three times alternating evacuated and flushed with argon. Palladium on activated charcoal, 10% Pd basis (136 mg, 127.8 μmol, 0.043 eq) was added carefully. The reaction flask was evacuated, flushed with argon, evacuated and flushed with hydrogen. The reaction mixture was stirred under hydrogen atmosphere (balloon) at room temperature for 3 h. Afterwards, the reaction mixture was filtered and rinsed with ethyl acetate/methanol. The filtrate was concentrated in vacuo to afford the title compound (929 mg, 97% yield) as light brown solid. LCMS: m/z 307.2 [M+H]⁺, ESI pos. The compound was used without further purification.

### Step H: (1R,2R)-2-[(1-Chloropyrido[3,4-d]pyridazin-4-yl)amino]cyclohexanol

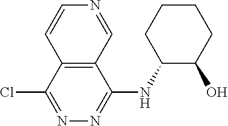 [SMILES: O[C@@H]1CCCC[C@H]1Nc1nnc(Cl)c2ccncc12]

To a mixture of (1R,2R)-2-aminocyclohexanol; hydrochloride (CAS #13374-31-7, 519 mg, 3.42 mmol, 1.30 eq) in 1,4-dioxane (2.5 mL) and water (0.28 mL) was added N,N-diisopropylethylamine (CAS #7087-68-5, 1.29 g, 1.7 mL, 10. mmol, 3.81 eq) followed by 1,4-dichloropyrido[3,4-d]pyridazine (CAS #14490-19-8, 525 mg, 2.62 mmol, 1.00 eq). The reaction mixture was stirred at 100 °C for 16 hours. The reaction mixture was cooled to room temperature and then extracted with ethyl acetate and half-saturated aq. NaHCO₃-solution. The aqueous layer was backextracted twice with ethyl acetate. The organic layers were washed with brine. The combined organic layers were dried over sodium sulfate, filtered and concentrated in vacuo. The crude product was adsorbed on ISOLUTE HM-N and purified by flash chromatography (silica gel, 40 g, gradient 0% to 5% methanol in dichloromethane) to afford a mixture of regioisomers that was submitted for further purification by SFC (column: IK; 12 nm, 5 μm, 250×20 mm; eluent B: 30% methanol+0.2% diethylamine) to afford the title compound (321 mg, 42% yield) as a light yellow solid. LCMS: m/z 279.2 [M+H]⁺, ESI pos.

### Step I: 5-[4-[[(1R,2R)-2-Hydroxycyclohexyl]amino]pyrido[3,4-d]pyridazin-1-yl]-6-(methoxymethyl)-2,3-dihydrobenzofuran-4-ol

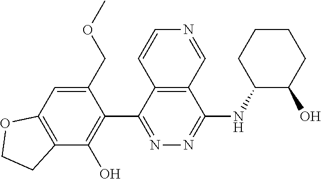 [SMILES: COCc1cc2c(c(O)c1-c1nnc(N[C@@H]3CCCC[C@H]3O)c3cnccc13)CCO2]

A mixture of (1R,2R)-2-[(1-chloropyrido[3,4-d]pyridazin-4-yl)amino]cyclohexanol (Example 8, step H) (60 mg, 0.20 mmol, 1.00 eq), 6-(methoxymethyl)-5-(4,4,5,5-tetramethyl-1,3,2-dioxaborolan-2-yl)-2,3-dihydrobenzofuran-4-ol (Example 8, step G) (110 mg, 0.34 mmol, 1.67 eq), potassium carbonate (100 mg, 0.72 mmol, 3.54 eq) and 1,1′-bis(diphenylphosphino)ferrocene-palladium(ii) dichloride dichloromethane complex (CAS #95464-05-4, 26 mg, 0.03 mmol, 0.16 eq) in 1,4-dioxane (1.4 mL) and water (0.70 mL) was flushed with argon and stirred at 95 °C for 16 hours. The reaction mixture was cooled to room temperature and extracted with ethyl acetate and half-saturated aq. NH₄Cl-solution. The aqueous layer was backextracted with ethyl acetate. The organic layers were washed with water and brine. The combined organic layers were dried over sodium sulfate, filtered and concentrated in vacuo. The crude product was adsorbed on ISOLUTE HM-N and purified by flash chromatography flash chromatography (Si-amine, 12 g, gradient 0% to 20% methanol in ethyl acetate) to afford the title compound (71 mg, 78% yield) as a light brown foam and as a mixture of atropisomers. LCMS: m/z 423.5 [M+H]⁺, ESI pos (for both atropisomers).

### Example 9

### trans-5-[4-[(4-Hydroxycyclohexyl)amino]pyrido[3,4-d]pyridazin-1-yl]-2,3-dihydrobenzofuran-4-ol

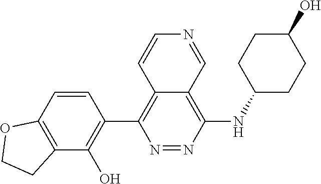 [SMILES: Oc1c(-c2nnc(N[C@H]3CC[C@H](O)CC3)c3cnccc23)ccc2c1CCO2]

### Step A: 4-[(1-chloropyrido[3,4-d]pyridazin-4-yl)amino]cyclohexanol

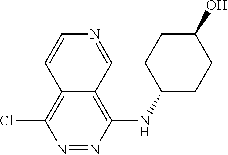 [SMILES: O[C@H]1CC[C@H](Nc2nnc(Cl)c3ccncc23)CC1]

To a mixture of trans-4-aminocyclohexanol (297 mg, 2.58 mmol, 1.3 eq) in 1,4-dioxane (1.8 mL) and water (0.180 mL) was added n,n-diisopropylethylamine (760 mg, 1 mL, 5.88 mmol, 3.0 eq) followed by 1,4-dichloropyrido[3,4-d]pyridazine (390 mg, 1.95 mmol, 1.0 eq). The reaction mixture was stirred at 100 °C for 18 hours.

The reaction mixture was cooled to room temperature and then extracted with ethyl acetate and half-saturated aqueous NaHCO₃-solution. The aqueous layer was backextracted twice with ethyl acetate. The organic layers were washed with brine. The combined organic layers were dried over sodium sulfate, filtered and concentrated in vacuo.

The crude product was purified by column chromatography on silica gel (25 g, gradient 0% to 10% methanol in ethyl acetate) to afford a mixture of regioisomers that was submitted for further purification via SFC (column achiral 4EPI, 12 nm, 5 μm, 250×20 mm; 15% MeOH) to afford the title compound (120 mg, 21% yield) as a light yellow solid.

MS: 279.1[M+H]⁺, ESI pos.

### StepB: trans-5-[4-[(4-Hydroxycyclohexyl)amino]pyrido[3,4-d]pyridazin-1-yl]-2,3-dihydrobenzofuran-4-ol

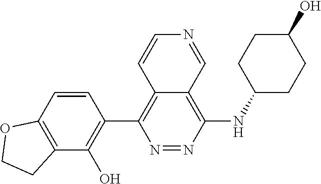 [SMILES: Oc1c(-c2nnc(N[C@H]3CC[C@H](O)CC3)c3cnccc23)ccc2c1CCO2]

In a sealable tube, a mixture of trans-4-[(1-chloropyrido[3,4-d]pyridazin-4-yl)amino]cyclohexanol (100 mg, 0.36 mmol, 1.000 eq), 5-(4,4,5,5-tetramethyl-1,3,2-dioxaborolan-2-yl)coumaran-4-ol (132 mg, 0.50 mmol, 1.40 eq) and cesium carbonate (351 mg, 1.08 mmol, 3.00 eq) in 1,4-dioxane (2.2 mL) and water (0.54 mL) was set under argon, and xphos-pd-g3 gt (46 mg, 0.054 mmol, 0.15 eq) was added finally. The reaction mixture was stirred at 90 °C in the sealed tube for 2 hours. The reaction mixture was cooled to room temperature. Quenched with water (15 mL) and sat NH₄Cl sol (3 mL), then extracted with ethyl acetate (2×40 mL). The organic layers were washed with brine (40 mL), dried over Na₂SO₄, filtered off and concentrated in vacuo.

The crude product was purified by column chromatography on silica gel (25 g, gradient 0% to 70% ethanol in ethyl acetate) to afford the title compound (46 mg, 32% yield) as a light yellow solid. MS: 379.2 [M+H]⁺, ESI pos.

### Example 10

### 5-[4-[[(1R,2R)-2-Hydroxycyclohexyl]amino]pyrido[3,4-d]pyridazin-1-yl]-6-methyl-2,3-dihydrobenzofuran-4-ol

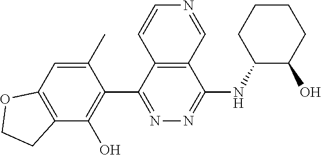 [SMILES: Cc1cc2c(c(O)c1-c1nnc(N[C@@H]3CCCC[C@H]3O)c3cnccc13)CCO2]

A mixture of (1R,2R)-2-[(1-chloropyrido[3,4-d]pyridazin-4-yl)amino]cyclohexanol (Example 8, step H) (120 mg, 0.41 mmol, 1.00 eq), 6-methyl-5-(4,4,5,5-tetramethyl-1,3,2-dioxaborolan-2-yl)-2,3-dihydrobenzofuran-4-ol (CAS #2923540-54-7, 203 mg, 0.66 mmol, 1.62 eq, 90% purity), potassium carbonate (200 mg, 1.45 mmol, 3.538 eq) and 1,1′-bis(diphenylphosphino)ferrocene-palladium(ii) dichloride dichloromethane complex (CAS #95464-05-4, 52 mg, 0.06 mmol, 0.16 eq) in 1,4-dioxane (2.8 mL) and water (1.4 mL) was flushed with argon and stirred at 95 °C for 16 hours. The reaction mixture was cooled to room temperature and extracted with ethyl acetate and half-saturated aq. NH₄Cl-solution. The aqueous layer was backextracted with ethyl acetate. The organic layers were washed with water and brine. The combined organic layers were dried over sodium sulfate, filtered and concentrated in vacuo. The crude product was adsorbed on ISOLUTE HM-N and purified by flash chromatography (silica gel, 12 g, gradient 0% to 10% methanol in dichloromethane). All fractions containing product were combined and concentrated in vacuo. The residue was repurified by flash chromatography (Si-amine, 12 g, gradient 0% to 10% methanol in ethyl acetate) to afford the title compound (96 mg, 57% yield) as a yellow solid and as mixture of atropisomers. LCMS: m/z 393.3 [M+H]⁺, ESI pos (for both atropisomers).

### Example 11

### cis-5-[4-[(4-Hydroxycyclohexyl)amino]pyrido[3,4-d]pyridazin-1-yl]-2,3-dihydrobenzo-furan-4-ol

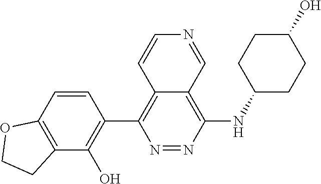 [SMILES: Oc1c(-c2nnc(N[C@H]3CC[C@@H](O)CC3)c3cnccc23)ccc2c1CCO2]

### Step A: 4-[(1-chloropyrido[3,4-d]pyridazin-4-yl)amino]cyclohexanol

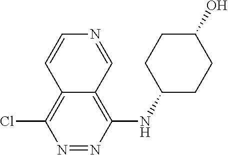 [SMILES: O[C@H]1CC[C@@H](Nc2nnc(Cl)c3ccncc23)CC1]

To a mixture of cis-4-aminocyclohexanol (297 mg, 2.58 mmol, 1.32 eq) in 1,4-dioxane (1.8 mL) and water (0.180 mL) was added n,n-diisopropylethylamine (760 mg, 1 mL, 5.88 mmol, 3.0 eq) followed by 1,4-dichloropyrido[3,4-d]pyridazine (390 mg, 1.95 mmol, 1.000 eq). The reaction mixture was stirred at 100 °C for 19 hours. The reaction mixture was cooled to room temperature and then extracted with ethyl acetate and half-saturated aq. NaHCO₃-solution. The aqueous layer was backextracted twice with ethyl acetate. The organic layers were washed with brine. The combined organic layers were dried over sodium sulfate, filtered and concentrated in vacuo. The crude product was purified by column chromatography on silica gel (25 g, gradient 0% to 10% methanol in ethyl acetate) to afford a mixture of regioisomers that was submitted for further purification via SFC (column achiral 4EPI, 12 nm, 5 μm, 250×20 mm; 10% MeOH) to afford the title compound (222 mg, 33% yield) as a light yellow solid.

MS: 279.2 [M+H]⁺, ESI pos.

### Step B: cis-5-[4-[(4-Hydroxycyclohexyl)amino]pyrido[3,4-d]pyridazin-1-yl]-2,3-dihydrobenzofuran-4-ol

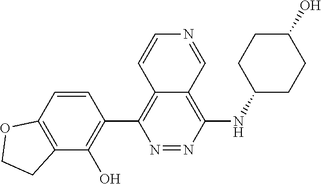 [SMILES: Oc1c(-c2nnc(N[C@H]3CC[C@@H](O)CC3)c3cnccc23)ccc2c1CCO2]

In a sealable tube, a mixture of cis-4-[(1-chloropyrido[3,4-d]pyridazin-4-yl)amino]cyclohexanol (100 mg, 0.36 umol, 1.0 eq), 5-(4,4,5,5-tetramethyl-1,3,2-dioxaborolan-2-yl)coumaran-4-ol (132 mg, 0.50 mmol, 1.40 eq) and cesium carbonate (351 mg, 1.08 mmol, 3.0 eq) in 1,4-dioxane (2.2 mL) and water (0.54 mL) was set under argon, and xphos-pd-g3 (46 mg, 0.053 mmol, 0.150 eq) was added finally. The reaction mixture was stirred at 90 °C in the sealed tube for 2 hours.

The reaction mixture was cooled to room temperature. Quenched with water (15 mL) and sat NH₄Cl sol (3 mL), then extracted with ethyl acetate (2×40 mL). The organic layers were washed with brine (40 mL), dried over Na₂SO₄, filtered off and concentrated in vacuo.

The crude product was purified by column chromatography on silica gel (25 g, gradient 0% to 70% ethanol in ethyl acetate) to afford the title compound (100 mg, 66% yield) as a light yellow solid. MS: 379.2 [M+H]⁺, ESI pos

### Example 13

### (1S,3R)-3-[[1-(4-Hydroxy-2,3-dihydrobenzofuran-5-yl)pyrido[3,4-d]pyridazin-4-yl]amino]-N-methyl-cyclohexanecarboxamide

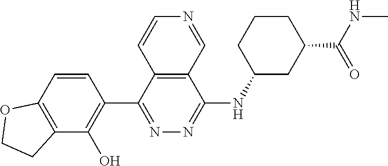 [SMILES: CNC(=O)[C@H]1CCC[C@@H](Nc2nnc(-c3ccc4c(c3O)CCO4)c3ccncc23)C1]

### Step A: cis-Methyl (1S,3R)-3-aminocyclohexanecarboxylate; hydrochloride

cis-(1S,3R)-3-(Tert-butoxycarbonylamino)cyclohexanecarboxylic acid (CAS #222530-34-9, 2.0 g, 7.97 mmol, 1.0 eq) was dissolved in dichloromethane (90 mL) and methanol (30 mL). Then 4 M HCl in 1,4-dioxane (16.0 mL, 63.8 mmol, 8.0 eq) was added to the reaction mixture, and it was stirred under argon for 16 h. The reaction solution was evaporated to dryness to afford the title compound (1.64 g, quant. yield) as a white solid. LCMS: m/z 158.1 [M+H]⁺, ESI pos.

### Step B: cis-Methyl (1S,3R)-3-[(1-chloropyrido[3,4-d]pyridazin-4-yl)amino]cyclo-hexanecarboxylate

To a mixture of cis-methyl (1S,3R)-3-aminocyclohexanecarboxylate; hydrochloride (770.5 mg, 3.78 mmol, 1.20 eq) in 1,4-dioxane (8.61 mL) and water (2.15 mL) was added at ambient temperature N,N-diisopropylethylamine (1.63 g, 2.14 mL, 12.6 mmol, 4.0 eq). After stirring for 5 minutes, 1,4-dichloropyrido[3,4-d]pyridazine (630 mg, 3.15 mmol, 1.0 eq) was added in one portion as a solid, resulting in a greenish solution. The reaction mixture was then stirred at 100 °C for 60 hours. Afterwards, the reaction mixture was quenched with semi saturated aq. NaHCO₃ solution (100 mL) and extracted with ethyl acetate (2×50 mL). The organic layers were washed with water (10 mL) and brine (10 mL). The combined organic extracts were dried over sodium sulfate, filtered and the solvent was evaporated under reduced pressure. The residue was purified by flash chromatography over silica gel (gradient 0-30% ethyl acetate in heptane) to afford a mixture of the two regioisomers, the title compound and methyl (1S,3R)-3-[(4-chloropyrido[3,4-d]pyridazin-1-yl)amino]cyclohexanecarboxylate (793 mg, 77%) as yellow solid. LCMS m/z: 321.1[M+H]⁺, ESI pos.

### Step C: cis-Methyl (1S,3R)-3-[[1-(4-hydroxy-2,3-dihydrobenzofuran-5-yl)pyrido[3,4-d]pyridazin-4-yl]amino]cyclohexanecarboxylate

In a sealable tube, a mixture of aforementioned cis-methyl (1S,3R)-3-[(1-chloropyrido[3,4-d]pyridazin-4-yl)amino]cyclo-hexanecarboxylate (700 mg, 1.96 mmol, 1.0 eq), 5-(4,4,5,5-tetramethyl-1,3,2-dioxaborolan-2-yl)coumaran-4-ol (800.8 mg, 2.75 mmol, 1.40 eq) and cesium carbonate (1.92 g, 5.89 mmol, 3.00 eq) in 1,4-dioxane (11.7 mL) and water (2.82 mL) was set under argon, and XPhos-Pd-G3 gt (249.36 mg, 294.59 μmol, 0.15 eq) was added. The reaction mixture was stirred at 90 °C in the sealed tube for 2 hours. Then the reaction mixture was cooled to room temperature, quenched with water (50 mL) and sat. aq. NH₄Cl solution (10 mL) and then extracted with ethyl acetate (2×50 mL). The organic layers were washed with brine (20 mL), dried over Na₂SO₄, filtered and concentrated in vacuo. The crude was purified by flash chromatography on silica gel (gradient 0% to 30% EtOH in ethyl acetate) to afford a mixture of regio-isomers (910 mg, 108%) as yellow solid. LCMS m/z: 421.2 [M+H]⁺, ESI pos. The regio-isomers were separated by SFC (column chiral IB, 5 μm, 250×20 mm, CO₂/MeOH+0.2% diethylamine) to afford the title compound (535 mg. 56% yield) as a yellow solid and cis-methyl (1R,3S,3-[[4-(4-hydroxy-2,3-dihydrobenzofuran-5-yl)pyrido[3,4-d]pyridazin-1-yl]amino]cyclohexanecarboxylate (100 mg, 11%) as green solid.

### Step D: cis-(1S,3R)-3-[[1-(4-hydroxy-2,3-dihydrobenzofuran-5-yl)pyrido[3,4-d]pyridazin-4-yl]amino]cyclohexanecarboxylic acid

To a solution of aforementioned cis-methyl (1S,3R)-3-[[1-(4-hydroxy-2,3-dihydrobenzofuran-5-yl)pyrido[3,4-d]pyridazin-4-yl]amino]cyclohexanecarboxylate (530 mg, 1.26 mmol, 1.0 eq) in methanol (3 mL) and tetrahydrofuran (6 mL) was added 1 M aq. LiOH solution (3.78 mL, 3.78 mmol, 3.0 eq) dropwise at ambient temperature. The yellow reaction solution was then stirred at 23 °C for 3 hours. The reaction mixture was neutralized with 5% citric acid and set to pH ~3-4 and extracted with ethyl acetate (2×30 mL). The organic layers were washed with brine (10 mL), dried over Na₂SO₄, filtered and concentrated in vacuo to afford the title compound (230 mg, 43% yield) as a light brown solid. LCMS m/z: 407.2 [M+H]⁺, ESI pos.

### Step E: (1S,3R)-3-[[1-(4-Hydroxy-2,3-dihydrobenzofuran-5-yl)pyrido[3,4-d]pyridazin-4-yl]amino]-N-methyl-cyclohexanecarboxamide

To a solution of cis-(1S,3R)-3-[[1-(4-hydroxy-2,3-dihydrobenzofuran-5-yl)pyrido[3,4-d]pyridazin-4-yl]amino]cyclohexanecarboxylic acid (44 mg, 105 μmol, 1.0 eq) in N,N-dimethylformamide (338 μL) was added at ambient temperature N,N-diisopropylethylamine (67.86 mg, 89.3 μL, 525 μmol, 5.0 eq) followed by HATU (61.74 mg, 157.5 μmol, 1.5 eq). The yellow solution was stirred for 5 min, then methylamine hydrochloride (12.8 mg, 189 μmol, 1.8 eq) was added. The reaction mixture was stirred at 23 °C for 16 hours. HATU (61.74 mg, 158 μmol, 1.5 eq), N,N-diisopropylethylamine (67.9 mg, 89.3 μL, 525 μmol, 5.0 eq) and methylamine hydrochloride (12.8 mg, 189 μmol, 1.8 eq) were added to the reaction, and it was stirred for 2 days. The reaction mixture was then quenched with water (2 mL) and extracted with ethyl acetate (2×5 mL). The organic layers were washed with brine (2 mL), dried over Na₂SO₄, filtered and concentrated in vacuo. The crude was purified by reverse phase (C18, RediSep Rf GOLD, gradient water/MeCN 0-40% MeCN) to afford the title compound (5.2 mg, 11% yield) as yellow solid. LCMS m/z: 420.2 [M+H]⁺, ESI pos.

### Example 14A

### 5-[4-[[(3S,4R)-3-Hydroxytetrahydropyran-4-yl]amino]pyrido[3,4-d]pyridazin-1-yl]-2,3-dihydrobenzofuran-4-ol or 5-[4-[[(3R,4S)-3-hydroxytetrahydropyran-4-yl]amino]pyrido[3,4-d]pyridazin-1-yl]-2,3-dihydrobenzofuran-4-ol

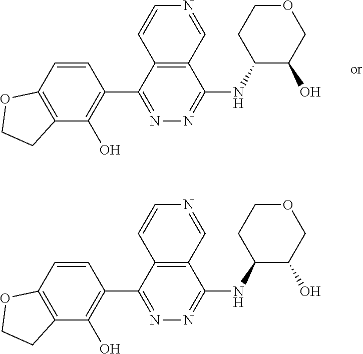 [SMILES: Oc1c(-c2nnc(N[C@@H]3CCOC[C@H]3O)c3cnccc23)ccc2c1CCO2 ; Oc1c(-c2nnc(N[C@H]3CCOC[C@@H]3O)c3cnccc23)ccc2c1CCO2]

### Step A: rac-(3S,4R)-4-[(1-Chloropyrido[3,4-d]pyridazin-4-yl)amino]tetrahydropyran-3-ol

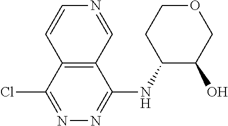 [SMILES: O[C@@H]1COCC[C@H]1Nc1nnc(Cl)c2ccncc12]

To a mixture of rac-(3S,4R)-4-aminotetrahydropyran-3-ol (CAS #215940-92-4, 200 mg, 1.71 mmol, 1.22 eq) in 1,4-dioxane (1.8 mL) and water (0.18 mL) was added N,N-diisopropylethylamine (CAS #7087-68-5, 547 mg, 0.72 mL, 4.23 mmol, 3.02 eq) followed by 1,4-dichloropyrido[3,4-d]pyridazine (CAS #14490-19-8, 280 mg, 1.40 mmol, 1.00 eq). The reaction mixture was stirred at 100 °C for 16 hours. The reaction mixture was cooled to room temperature and then extracted with ethyl acetate and half-saturated aq. NaHCO₃-solution. The aqueous layer was backextracted with ethyl acetate. The organic layers were washed with brine. The combined organic layers were dried over sodium sulfate, filtered and concentrated in vacuo. The crude product was adsorbed on ISOLUTE HM-N and purified by flash chromatography (silica gel, 25 g, gradient 0% to 10% methanol in dichloromethane) to afford a mixture of regioisomers that was submitted for further purification by SFC (column: achiral column DEA, eluent B: 15% methanol+0.2% diethylamine) to afford the title compound (165 mg, 40% yield) as a light yellow solid (first eluting, retention time=3.33 minutes). LCMS: m/z 281.1 [M+H]⁺, ESI pos.

### Step B: 5-[4-[[rac-(3S,4R)-3-Hydroxytetrahydropyran-4-yl]amino]pyrido[3,4-d]pyridazin-1-yl]-2,3-dihydrobenzofuran-4-ol

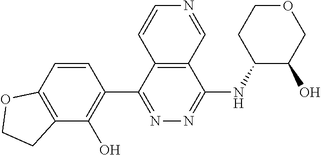 [SMILES: Oc1c(-c2nnc(N[C@@H]3CCOC[C@H]3O)c3cnccc23)ccc2c1CCO2]

A mixture of rac-(3S,4R)-4-[(1-chloropyrido[3,4-d]pyridazin-4-yl)amino]tetrahydropyran-3-ol_(Example 14A, step A) (148 mg, 0.50 mmol, 1.00 eq), 5-(4,4,5,5-tetramethyl-1,3,2-dioxaborolan-2-yl)-2,3-dihydrobenzofuran-4-ol (CAS #2923540-32-1, 240 mg, 0.87 mmol, 1.74 eq), potassium carbonate (245 mg, 1.77 mmol, 3.54 eq) and 1,1′-bis(diphenylphosphino)ferrocene-palladium(ii) dichloride dichloromethane complex (CAS #95464-05-4, 64 mg, 0.08 mmol, 0.16 eq) in 1,4-dioxane (3.4 mL) and water (1.7 mL) was flushed with argon and stirred at 95 °C for 16 hours. The reaction mixture was cooled to room temperature and extracted with ethyl acetate and half-saturated aq. NH₄Cl-solution. The aqueous layer was backextracted with ethyl acetate. The organic layers were washed with water and brine. The combined organic layers were dried over sodium sulfate, filtered and concentrated in vacuo. The crude product was adsorbed on ISOLUTE HM-N and purified by flash chromatography (silica gel, 12 g, gradient 0% to 10% methanol in dichloromethane) to afford the title compound (173 mg, 86% yield) as a brown foam. LCMS: m/z 381.2 [M+H]⁺, ESI pos.

### Step C: 5-[4-[[(3S,4R)-3-Hydroxytetrahydropyran-4-yl]amino]pyrido[3,4-d]pyridazin-1-yl]-2,3-dihydrobenzofuran-4-ol or 5-[4-[[(3R,4S)-3-hydroxytetrahydropyran-4-yl]amino]pyrido[3,4-d]pyridazin-1-yl]-2,3-dihydrobenzofuran-4-ol

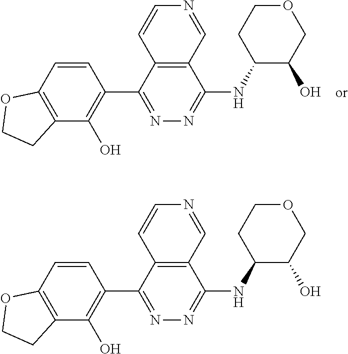 [SMILES: Oc1c(-c2nnc(N[C@@H]3CCOC[C@H]3O)c3cnccc23)ccc2c1CCO2 ; Oc1c(-c2nnc(N[C@H]3CCOC[C@@H]3O)c3cnccc23)ccc2c1CCO2]

Chiral separation of 5-[4-[[rac-(3S,4R)-3-hydroxytetrahydropyran-4-yl]amino]pyrido[3,4-d]pyridazin-1-yl]-2,3-dihydrobenzofuran-4-ol (Example 14A, step B) (172 mg, 0.43 mmol, 1.00 eq) by SFC (column: chiral OJ, eluent B: 30% methanol+0.2% diethylamine) to afford the title compound (65 mg, 38% yield) as a yellow foam (second eluting, retention time=3.03 minutes). LCMS: m/z 381.2 [M+H]⁺, ESI pos.

### Example 14B

### 5-[4-[[(3R,4S)-3-Hydroxytetrahydropyran-4-yl]amino]pyrido[3,4-d]pyridazin-1-yl]-2,3-dihydrobenzofuran-4-ol or 5-[4-[[(3S,4R)-3-hydroxytetrahydropyran-4-yl]amino]pyrido[3,4-d]pyridazin-1-yl]-2,3-dihydrobenzofuran-4-ol

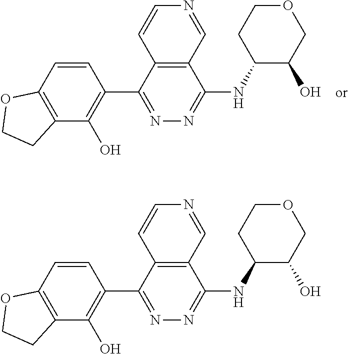 [SMILES: Oc1c(-c2nnc(N[C@@H]3CCOC[C@H]3O)c3cnccc23)ccc2c1CCO2 ; Oc1c(-c2nnc(N[C@H]3CCOC[C@@H]3O)c3cnccc23)ccc2c1CCO2]

Chiral separation of 5-[4-[[rac-(3S,4R)-3-hydroxytetrahydropyran-4-yl]amino]pyrido[3,4-d]pyridazin-1-yl]-2,3-dihydrobenzofuran-4-ol (Example 14A, step B) (172 mg, 0.43 mmol, 1.00 eq) by SFC (column: chiral OJ, eluent B: 30% methanol+0.2% diethylamine) to afford the title compound (66 mg, 38% yield) as a yellow foam (first eluting, retention time=2.33 minutes). LCMS: m/z 381.2 [M+H]⁺, ESI pos.

### Example 15

### 5-[4-[[(3R,4R)-3-Hydroxytetrahydropyran-4-yl]amino]pyrido[3,4-d]pyridazin-1-yl]-2,3-dihydrobenzofuran-4-ol

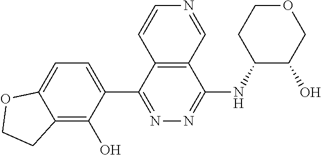 [SMILES: Oc1c(-c2nnc(N[C@@H]3CCOC[C@@H]3O)c3cnccc23)ccc2c1CCO2]

### Step A: (3R,4R)-4-[(1-Chloropyrido[3,4-d]pyridazin-4-yl)amino]tetrahydropyran-3-ol

To a mixture of 1,4-dichloropyrido[3,4-d]pyridazine (300 mg, 1.42 mmol, 1.0 eq; CAS #14490-19-8) and (3R,4R)-4-aminotetrahydropyran-3-ol; hydrochloride (331 mg, 2.15 mmol, 1.51 eq; CAS #1523530-38-2) in 1,4-dioxane (1.8 mL) was added N,N-diisopropylethylamine (555 mg, 750 μL, 4.3 mmol, 3.0 eq). The reaction mixture was then stirred at 100 °C for two hours. The reaction mixture was extracted with ethyl acetate (~40 mL) and water (~5 mL). The aqueous layer was backextracted with ethyl acetate (~40 mL). The organic layers were washed with water (~5 mL) and brine (~5 mL). The combined organic layers were dried over sodium sulfate, filtered and concentrated in vacuo. The crude was purified by flash chromatography on silica gel (gradient 0% to 10% MeOH in DCM) to afford the title compound and ((3R,4R)-4-[(4-chloropyrido[3,4-d]pyridazin-1-yl)amino]tetrahydropyran-3-ol as a 1:0.4 mixture of region-isomers as yellow solid (448 mg, 51% yield). LCMS m/z: 281.1[M+H]⁺, ESI pos.

### Step B: 5-[4-[[(3R,4R)-3-Hydroxytetrahydropyran-4-yl]amino]pyrido[3,4-d]pyridazin-1-yl]-2,3-dihydrobenzofuran-4-ol

A mixture of 5-(4,4,5,5-tetramethyl-1,3,2-dioxaborolan-2-yl)coumaran-4-ol (155.6 mg, 534.4 μmol, 1.50 eq, CAS #2923540-32-1), aforementioned (3R,4R)-4-[(1-chloropyrido[3,4-d]pyridazin-4-yl)amino]tetrahydropyran-3-ol (200 mg, 356.2 μmol, 1.0 eq) and cesium carbonate (348.2 mg, 1.1 mmol, 3.0 eq) in water (0.55 mL) and 1,4-dioxane (2.2 mL) was flushed with argon. XPhos Pd G3 (45.2 mg, 53.4 μmol, 0.15 eq) was added to the reaction mixture and stirred at 95 °C for two hours. The reaction mixture was cooled to room temperature and extracted with ethyl acetate and water. The aqueous layer was backextracted with ethyl acetate two times. The organic layers were washed with water and brine. The combined organic layers were dried over sodium sulfate, filtered and concentrated in vacuo. The crude was then purified by flash chromatography on silica gel (gradient 0% to 10% methanol in dichloromethane) to afford a mixture of regioisomers (110 mg). After preparative HPLC, the title compound was obtained as light brown solid (56 mg, 39% yield; LCMS m/z: 381.3 [M+H]⁺, ESI pos) and 5-[1-[[(3R,4R)-3-hydroxytetrahydropyran-4-yl]amino]pyrido[3,4-d]pyridazin-4-yl]coumaran-4-ol (35 mg, 26% yield) as light brown solid.

### Example A

A compound of formula I can be used in a manner known per se as the active ingredient for the production of tablets of the following composition:

| | Per tablet | |
|---|---|---|
| Active ingredient | 200 | mg |
| Microcrystalline cellulose | 155 | mg |
| Corn starch | 25 | mg |
| Talc | 25 | mg |
| Hydroxypropylmethylcellulose | 20 | mg |
| | 425 | mg |

### Example B

A compound of formula I can be used in a manner known per se as the active ingredient for the production of capsules of the following composition:

| | Per capsule | |
|---|---|---|
| Active ingredient | 100.0 | mg |
| Corn starch | 20.0 | mg |
| Lactose | 95.0 | mg |
| Talc | 4.5 | mg |
| Magnesium stearate | 0.5 | mg |
| | 220.0 | mg |

1. A compound of formula I 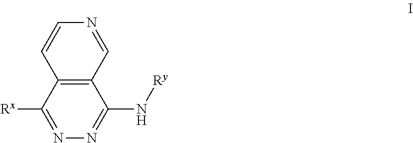
wherein,
R^{x} is selected from 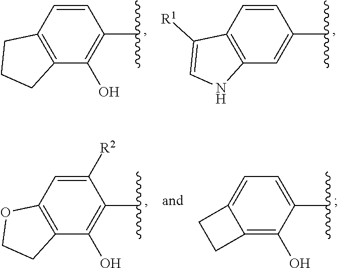
R¹ is H or halo;
R² is H, methyl, —CH₂OCH₃, or —CH₂OH;
R^{y} is selected from 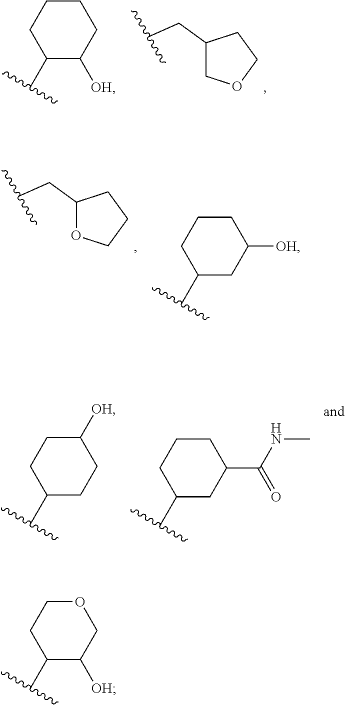
and pharmaceutically acceptable salts thereof.

2. A compound according to claim 1, wherein
R^{x} is selected from 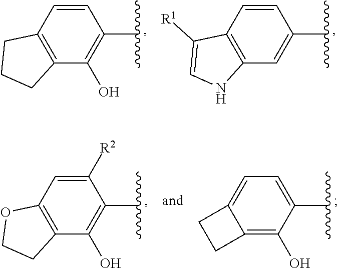
R¹ is H or halo;
R² is H, methyl or —CH₂OCH₃;
R^{y} is selected from 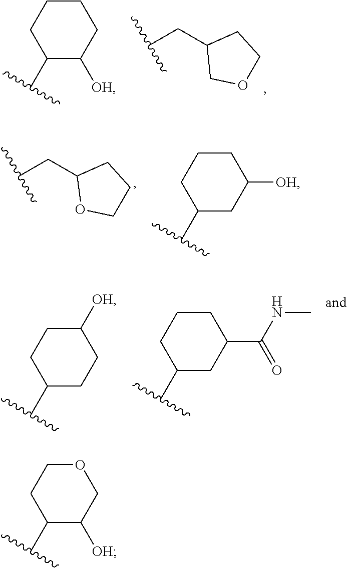
and pharmaceutically acceptable salts thereof.

3. A compound according to claim 1, wherein
R^{x} is 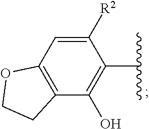
R² is H, methyl or —CH₂OCH₃;
R^{y} is selected from 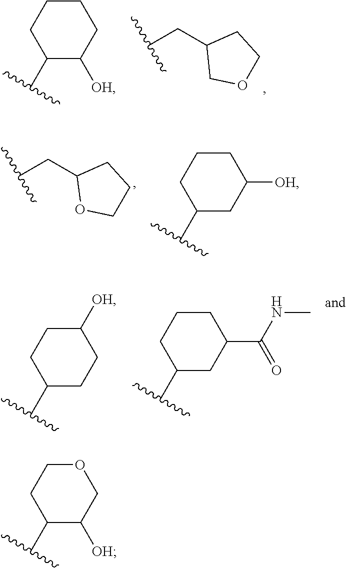
and pharmaceutically acceptable salts thereof.

4. A compound according to claim 1, wherein
R^{x} is 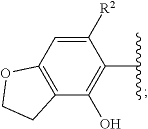
R² is H or —CH₂OCH₃;
R^{y} is selected from 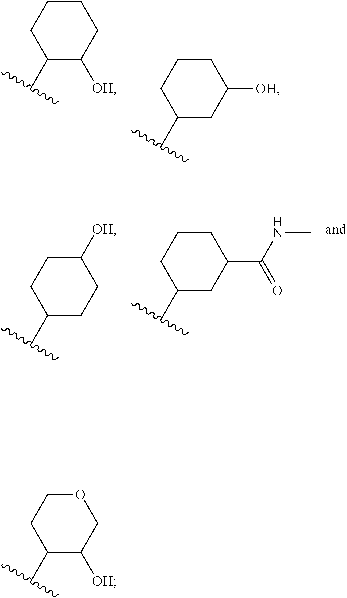 [SMILES: *C1CCCCC1O ; *C1CCCC(O)C1 ; *C1CCC(O)CC1 ; *C1CCCC(C(=O)NC)C1 ; *C1CCOCC1O]
and pharmaceutically acceptable salts thereof.

5. A compound according to claim 1, wherein
R^{x} is 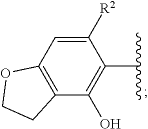
R² is H;
R^{y} is selected from 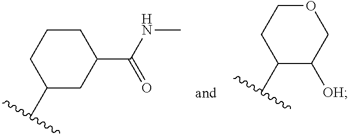 [SMILES: *C1CCCC(C(=O)NC)C1 ; *C1CCOCC1O]
and pharmaceutically acceptable salts thereof.

6. A compound according to claim 1, wherein
R^{x} is 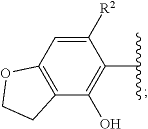
R² is —CH₂OCH₃;
R^{y} is selected from 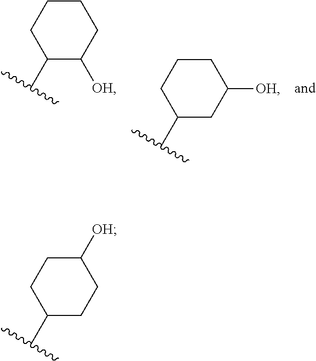 [SMILES: *C1CCCCC1O ; *C1CCCC(O)C1 ; *C1CCC(O)CC1]
and pharmaceutically acceptable salts thereof.

7. A compound according claim 1, selected from
5-[4-[[rac-(1R,2R)-2-Hydroxycyclohexyl]amino]pyrido[3,4-d]pyridazin-1-yl]indan-4-ol;
5-[4-[[rel-(1R,2R)-2-hydroxycyclohexyl]amino]pyrido[3,4-d]pyridazin-1-yl]indan-4-ol;
5-[4-[[rel-(1S,2S)-2-hydroxycyclohexyl]amino]pyrido[3,4-d]pyridazin-1-yl]indan-4-ol;
5-[4-[[rac-(1R,2R)-2-hydroxycyclohexyl]amino]pyrido[3,4-d]pyridazin-1-yl]-2,3-dihydrobenzofuran-4-ol;
5-[4-[[rel-(1R,2R)-2-Hydroxycyclohexyl]amino]pyrido[3,4-d]pyridazin-1-yl]-2,3-dihydrobenzofuran-4-ol;
5-[4-[[rel-(1S,2S)-2-Hydroxycyclohexyl]amino]pyrido[3,4-d]pyridazin-1-yl]-2,3-dihydrobenzofuran-4-ol;
rac-(1R,2R)-2-[[1-(1H-indol-6-yl)pyrido[3,4-d]pyridazin-4-yl]amino]cyclohexanol;
1-(3-Fluoro-1H-indol-6-yl)-N-[[rel-(3R)-tetrahydrofuran-3-yl]methyl]pyrido[3,4-d]pyridazin-4-amine;
1-(3-fluoro-1H-indol-6-yl)-N-[[rel-(3S)-tetrahydrofuran-3-yl]methyl]pyrido[3,4-d]pyridazin-4-amine;
5-[4-[[rac-(3S)-Tetrahydrofuran-3-yl]methylamino]pyrido[3,4-d]pyridazin-1-yl]-2,3-dihydrobenzofuran-4-ol;
5-[4-[[rel-(3R)-tetrahydrofuran-3-yl]methylamino]pyrido[3,4-d]pyridazin-1-yl]-2,3-dihydrobenzofuran-4-ol;
5-[4-[[rel-(3S)-tetrahydrofuran-3-yl]methylamino]pyrido[3,4-d]pyridazin-1-yl]-2,3-dihydrobenzofuran-4-ol;
5-[4-[[rel-(2R)-Tetrahydrofuran-2-yl]methylamino]pyrido[3,4-d]pyridazin-1-yl]-2,3-dihydrobenzofuran-4-ol;
5-[4-[[rel-(2S)-Tetrahydrofuran-2-yl]methylamino]pyrido[3,4-d]pyridazin-1-yl]-2,3-dihydrobenzofuran-4-ol;
3-[4-[[rac-(1R,2R)-2-Hydroxycyclohexyl]amino]pyrido[3,4-d]pyridazin-1-yl]bicyclo[4.2.0]octa-1,3,5-trien-2-ol
and pharmaceutically acceptable salts thereof.

8. A compound according to claim 1, selected from
5-[4-[[(1 R,2R)-2-Hydroxycyclohexyl]amino]pyrido[3,4-d]pyridazin-1-yl]-6-(methoxymethyl)-2,3-dihydrobenzofuran-4-ol;
trans 5-[4-[(4-Hydroxycyclohexyl)amino]pyrido[3,4-d]pyridazin-1-yl]-2,3-dihydrobenzofuran-4-ol;
5-[4-[[(1 R,2R)-2-Hydroxycyclohexyl]amino]pyrido[3,4-d]pyridazin-1-yl]-6-methyl-2,3-dihydrobenzofuran-4-ol;
cis 5-[4-[(4-Hydroxycyclohexyl)amino]pyrido[3,4-d]pyridazin-1-yl]-2,3-dihydrobenzofuran-4-ol;
5-[4-[[(1R,3S)-3-Hydroxycyclohexyl]amino]pyrido[3,4-d]pyridazin-1-yl]-2,3-dihydrobenzofuran-4-ol;
(1S,3R)-3-[[1-(4-Hydroxy-2,3-dihydrobenzofuran-5-yl)pyrido[3,4-d]pyridazin-4-yl]amino]-N-methyl-cyclohexanecarboxamide;
5-[4-[[(3R,4S)-3-Hydroxytetrahydropyran-4-yl]amino]pyrido[3,4-d]pyridazin-1-yl]-2,3-dihydrobenzofuran-4-ol;
5-[4-[[(3S,4R)-3-Hydroxytetrahydropyran-4-yl]amino]pyrido[3,4-d]pyridazin-1-yl]-2,3-dihydrobenzofuran-4-ol;
and pharmaceutically acceptable salts thereof.

9. A pharmaceutical composition comprising a compound according to claim 1 and a therapeutically inert carrier.

10-18. (canceled)

19. A method of inhibiting NLRP3, which method comprises administering an effective amount of a compound as claimed in claim 1 to inhibit NLRP3.

20. A method for the treatment or prophylaxis of a disease, disorder or condition, which method comprises administering an effective amount of a compound according to claim 1 wherein the disease, disorder or condition is selected from Asthma or COPD.

21. A method for the treatment or prophylaxis of a disease, disorder or condition, which method comprises administering an effective amount of a compound according to claim 1 wherein the disease, disorder or condition is selected from Parkinson's Disease or Alzheimer's Disease.

22. A method of inhibiting NLRP3, which method comprises administering an effective amount of a compound as claimed in claim 7 to inhibit NLRP3.

23. A method for the treatment or prophylaxis of a disease, disorder or condition, which method comprises administering an effective amount of a compound according to claim 7 wherein the disease, disorder or condition is selected from Asthma or COPD.

24. A method for the treatment or prophylaxis of a disease, disorder or condition, which method comprises administering an effective amount of a compound according to claim 7 wherein the disease, disorder or condition is selected from Parkinson's Disease or Alzheimer's Disease.

25. A method of inhibiting NLRP3, which method comprises administering an effective amount of a compound as claimed in claim 8 to inhibit NLRP3.

26. A method for the treatment or prophylaxis of a disease, disorder or condition, which method comprises administering an effective amount of a compound according to claim 8 wherein the disease, disorder or condition is selected from Asthma or COPD.

27. A method for the treatment or prophylaxis of a disease, disorder or condition, which method comprises administering an effective amount of a compound according to claim 8 wherein the disease, disorder or condition is selected from Parkinson's Disease or Alzheimer's Disease.
